(19) United States
(12) Patent Application Publication
(10) Pub. No.: US 2012/0158041 A1
(43) Pub. Date: Jun. 21, 2012

(54) TACTICAL PANTS

(76) Inventor: Richard Gene Craig, Holladay, UT (US)

(21) Appl. No.: 13/327,703

(22) Filed: Dec. 15, 2011

Related U.S. Application Data
(60) Provisional application No. 61/423,537, filed on Dec. 15, 2010.
(60) Provisional application No. 61/493,867, filed on Jun. 6, 2011.

(51) Int. Cl. A61B 17/00; F41H 1/02; A41D 1/06

(52) U.S. Cl. 606/203; 2/227; 2/2.5

(57) ABSTRACT

Tactical pants can include one or more features for retaining a utility object, such as a firearm, for protecting against ballistic projectiles, or for reducing the effects of leg wounds. Various pants can include one or more of a weight distribution system, an anti-ballistic shield, or one or more tourniquets.

### CROSS-REFERENCE TO RELATED APPLICATIONS

This application claims the benefit of U.S. Provisional Application No. 61/423,537, filed Dec. 15, 2010, titled PANTS WITH HOLSTER, TOURNIQUET AND BALLISTIC RESISTANCE, and also claims the benefit of U.S. Provisional Application No. 61/493,867, filed Jun. 6, 2011, titled TACTICAL PANTS, the entire contents of each of which are hereby incorporated by reference herein.

### TECHNICAL FIELD

The present disclosure relates to pants for use by individuals who may use a firearm and/or who may be exposed to ballistic projectiles.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a right side elevation view of an embodiment of pants that can include one or more features for retaining a utility object (such as a firearm), protecting against ballistic projectiles, and/or reducing the effects of wounds sustained from ballistic projectiles or the like, wherein an embodiment of a utility compartment and embodiments of tourniquet covers are each shown in a closed configuration.

FIG. 2 is a right side elevation view of the pants of FIG. 1, wherein the utility compartment and one of the tourniquet covers are each shown in an open configuration;

FIG. 3 is a front perspective view of the pants of FIG. 1 showing an embodiment of a weight distributing system, wherein outer pants material is shown in broken lines to provide a view of an interior of the pants;

FIG. 4 is a partial cross-sectional view of the pants of FIG. 1, taken along the view line 4-4 in FIG. 1, which provides a further view of the weight distributing system;

FIG. 5A is a partial perspective view of an interior region of the pants of FIG. 1, wherein an embodiment of a longitudinal strap system is shown in a decoupled state;

FIG. 5B is further partial perspective view of the interior region of the pants of FIG. 1, wherein the longitudinal strap system is shown in a coupled state;

FIG. 6 is a front perspective view of a portion of an embodiment of a utility module that includes two mounting brackets, which is compatible with the pants of FIG. 1, wherein the mounting brackets are shown in a coupled configuration;

FIG. 7 is a rear perspective view of the portion of the embodiment of a utility module of FIG. 6, wherein the mounting brackets are shown a coupled configuration;

FIG. 8 is a perspective view of one of the mounting brackets of FIG. 6 shown decoupled from the other mounting bracket;

FIG. 9 is a perspective view of the other mounting bracket of FIG. 6;

FIG. 10 is a perspective view of a further portion of an embodiment of a utility module, such as that of FIG. 6, wherein an embodiment of a holster is securely attached to a mounting bracket such as that shown in FIG. 9;

FIG. 11 is a perspective view of an embodiment of a firearm being positioned in the holster of FIG. 10;

FIG. 12 is a perspective view of a further portion of another embodiment of a utility module, such as that of FIG. 6, wherein an embodiment of a utility pouch is securely attached to a mounting bracket such as that shown in FIG. 9;

FIG. 13 is a perspective view of an embodiment of a lateral strap system, which is compatible with the pants of FIG. 1;

FIG. 14 is a perspective view of another embodiment of a lateral strap system, which is compatible with the pants of FIG. 1, wherein the lateral strap system includes a locking assembly;

FIG. 15 is a cross-sectional view of the lateral strap system of FIG. 14, taken along the view line 15-15 in FIG. 14;

FIG. 16 is a front elevation view of an embodiment of a ballistic-resistant shield that is configured to protect critical blood vessels of a wearer thereof;

FIG. 17 is a cross-sectional view of the ballistic-resistant shield of FIG. 16 and the wearer thereof, taken along the view line 17-17 of FIG. 16, which further illustrates the trajectory of a projectile toward a critical blood vessel;

FIG. 18 is a front perspective view of another embodiment of a ballistic-resistant shield that is configured to protect critical blood vessels of a wearer thereof;

FIG. 19 is a front perspective view of another embodiment of pants, such as the pants of FIG. 1, which include the ballistic-resistant shield of FIG. 18 incorporated therein;

FIG. 20 is a cross-sectional view of the pants of FIG. 19 taken along the view line 20-20 in FIG. 19, which further illustrates the trajectory of a projectile toward a critical blood vessel;

FIG. 21 is a front perspective view of another embodiment of a ballistic-resistant shield that is configured to protect critical blood vessels of a wearer thereof;

FIG. 22 is a front perspective view of another embodiment of pants, such as the pants of FIG. 1, which include the ballistic-resistant shield of FIG. 21 incorporated therein;

FIG. 23A is a partial cross-sectional view of the pants of FIG. 22 taken along the view line 23A-23A in FIG. 22;

FIG. 23B is a partial cross-sectional view of the ballistic-resistant shield of FIG. 21 taken along the view line 23B-23B in FIG. 21, which depicts an embodiment of a composite material;

FIG. 24 is a partial front elevation view of the ballistic-resistant shield of FIG. 21, which depicts a stitching pattern;

FIG. 25 is an enlarged view thereof taken along the view line 25 in FIG. 24;

FIG. 26 is a front perspective view of another embodiment of a ballistic-resistant shield that is configured to protect critical blood vessels of a wearer thereof, wherein the ballistic-resistant shield includes mobility seams that are configured to increase mobility of a wearer of the ballistic-resistant shield;

FIG. 27 is a partial cross-sectional view of the ballistic-resistant shield of FIG. 26 taken along the view line 27-27 in FIG. 26;

FIG. 28 is another partial cross-sectional view of the ballistic-resistant shield of FIG. 26 taken along the view line 28-28 in FIG. 26, which depicts a portion of an embodiment of a mobility seam;

FIG. 29 is a partial cross-sectional view of another embodiment of a ballistic-resistant shield, such as the view depicted in FIG. 28, which depicts a portion of another embodiment of a mobility seam;

FIG. 30 is a partial cross-sectional view of another embodiment of a ballistic-resistant shield, such as the view depicted in FIG. 28, which depicts a portion of another embodiment of a mobility seam;

FIG. 31 is a partial cross-sectional view of another embodiment of a ballistic-resistant shield, such as the view depicted in FIG. 28, which depicts a portion of another embodiment of a mobility seam;

FIG. 32 is a partial cross-sectional view of another embodiment of a ballistic-resistant shield, such as the view depicted in FIG. 28, which depicts a portion of another embodiment of a mobility seam

FIG. 33 is a partial cross-sectional view of another embodiment of a ballistic-resistant shield, such as the view depicted in FIG. 28, which depicts portions of additional embodiments of mobility seams;

FIG. 34 is a partial cross-sectional view of another embodiment of a ballistic-resistant shield, such as the view depicted in FIG. 28, which depicts portions of additional embodiments of mobility seams;

FIG. 35 is a front perspective view of another embodiment of a ballistic-resistant shield that includes kinetic pads;

FIG. 36 is a cross-sectional view of the ballistic-resistant shield and a kinetic pad taken along the view line 36-36 in FIG. 35, which further illustrates the trajectory of a projectile toward a critical blood vessel;

FIGS. 37A-37E are front perspective views of another embodiment of pants, such as the pants of FIG. 1, that includes a tourniquet member, wherein the various views illustrate various stages in a method of utilizing the tourniquet.

### DETAILED DESCRIPTION

Various embodiments of pants are disclosed herein, which can include one or more features for retaining a utility object (such as a firearm), protecting against ballistic projectiles, and/or reducing the effects of wounds sustained from ballistic projectiles or the like. The pants can be desirable in a variety of contexts, such as, for example, tactical or combat situations. Although discussion of many of the embodiments herein is provided in such tactical situations, it should be understood that the pants may be used in other environments. For example, the pants may be used by police or other government personnel who regularly carry firearms and/or who may be at a heightened risk of experiencing gunfire or other ballistic projectiles. Accordingly, use of the word “tactical” is by way of illustration, and is not intended to be limiting.

One of more of the features discussed herein may be used in various embodiments of pants or other garments. Some of the advantages of various features, are specifically discussed hereafter, while other or further advantages can be appreciated from the present disclosure.

For example, certain tactical pants can be worn over the lower torso and leg region of the wearer. In various embodiments, the tactical pants can have pockets that can be sized for holding a handgun holster or similarly sized utility object. Weight from the utility object can be transferred to the thigh and/or the waist of the wearer by a harness or weight distribution system, which can significantly limit the amount of weight that is transferred through the visible fabric of the pants. In some instances, such weight distribution systems can conceal from an observer that the wearer is carrying a weapon. In other or further instances, a weight distribution system can increase the comfort of carrying the weapon within the pants. In still other or further instances, the weight distribution can prevent wearing of the visible portions of the fabric.

In other or further embodiments, a ballistic protective armor, or ballistic-resistant shield, can also be coupled (e.g., fastened to or integrally attached) with the pants to provide the wearer significant protection from projectiles directed toward critical blood vessels. In other or further embodiments, the pants can include medical treatment devices integrated into the pants construction for readily treating traumatic injuries. For example, the pants can include one or more tourniquet apparatuses.

It will be readily understood that the components of the embodiments as generally described and illustrated in the figures herein could be arranged and designed in a wide variety of different configurations. Thus, the following more detailed description of various embodiments, as represented in the figures, is not intended to limit the scope of the disclosure, as claimed, but is merely representative of various embodiments. While the various aspects of the embodiments are presented in drawings, the drawings are not necessarily drawn to scale unless specifically indicated.

The phrases “connected to” and “coupled to” refer to any form of interaction between two or more entities, including mechanical, electrical, magnetic, electromagnetic, fluid, and thermal interaction. Two components may be coupled to each other even though they are not in direct contact with each other. Like reference numerals are used for like features throughout this disclosure.

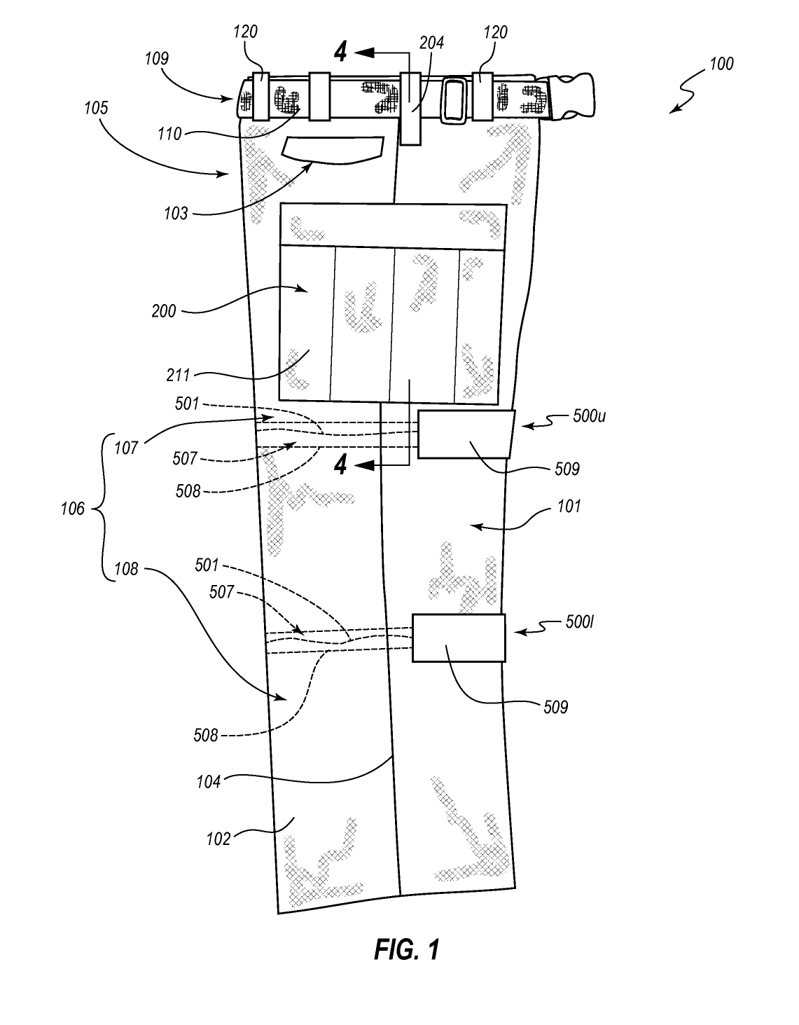

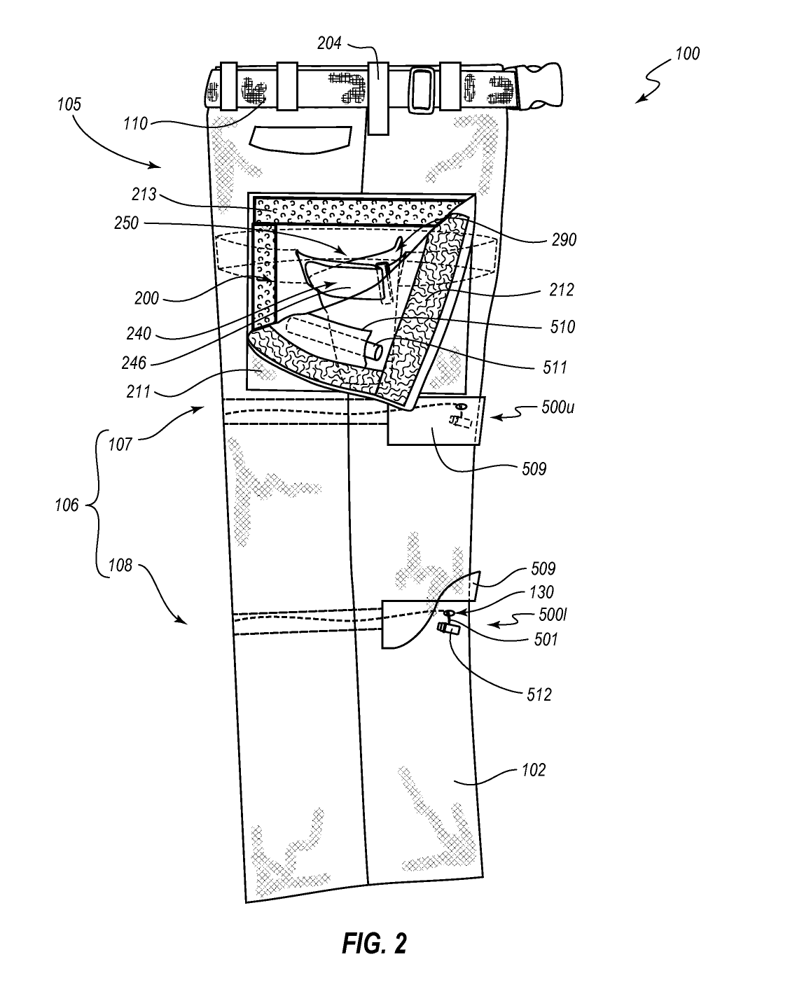

FIGS. 1 and 2 illustrate an embodiment of a pair of tactical pants 100. The pants 100 include an outer shell, outer layer, outer construction, or covering 101 that can have the appearance and/or construction of any suitable pants. For example, the covering 101 can resemble standard pant styles. The covering 101 may include one or more standard features, such as pockets 103. The covering can be constructed of any suitable pants material 102, such as combat pants material. Multiple pieces of the pants material 102 can be joined together in any suitable manner, such as via stitching, along seams 104. The material 102 can be flexible or otherwise capable of conforming to or permitting movement of a wearer of the pants 100.

The pants 100 can include a waist portion or waist region 105 that is configured to be worn around a waist of a wearer of the pants so as to support the pants thereon. The waist region 105 can be configured to hang on or support the pants 100 on the waist in any suitable manner. In some embodiments, in order to assist in maintaining the pants on the waist of the wearer, the pants 100 can include a plurality of belt loops 120 positioned along a waistline 109 of the pants 100. The belt loops 120 can be configured to receive a belt 110, which can secure the waistline 109 to the waist of the wearer and thus transfer the weight load of the pants 100 to the waist of the wearer. In other embodiments, suspenders (not shown) may be used to carry the weight load of the pants 100.

Two leg portions or leg regions 106 can extend downwardly from the waist region 105. When the pants 100 are worn, the waist region 105 can bear the weight of the leg regions 106. Each leg region 106 can include an upper leg portion 107 and a lower leg portion 108. The upper leg portion 107 can be configured to encompass an upper leg of a wearer (e.g., a thigh), and the lower leg portion 108 can be configured to encompass a lower leg of the wearer (e.g., a calf).

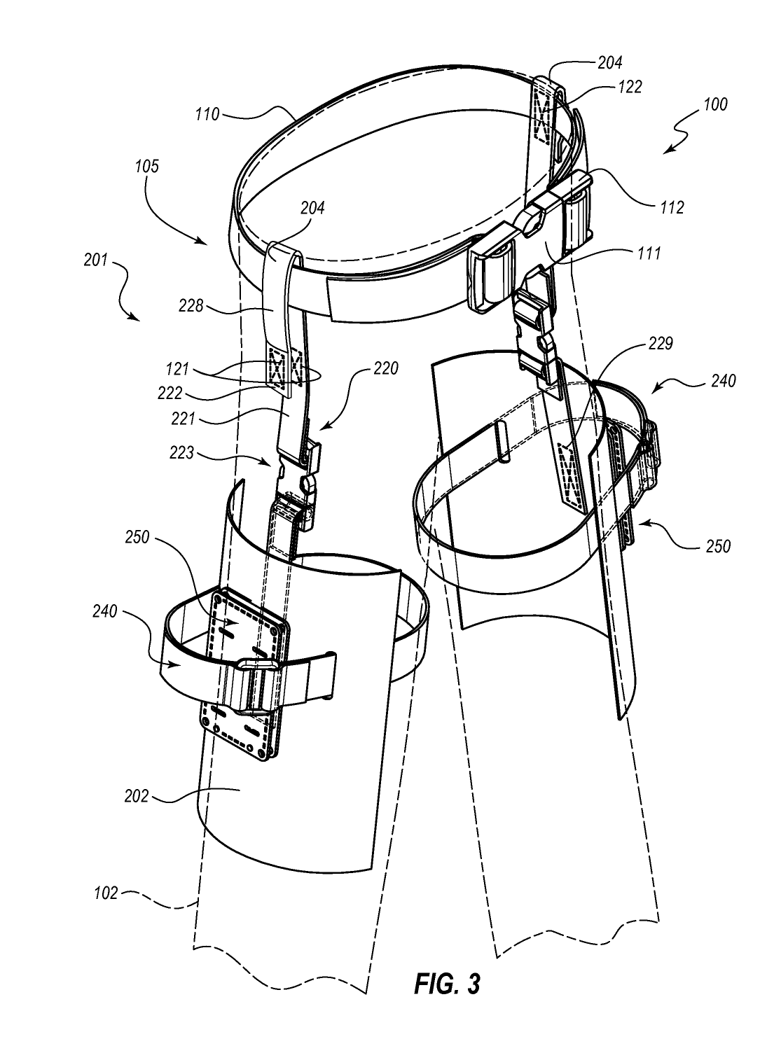

The covering 101 can include one or more releasable pockets 200. In the illustrated embodiment, the releasable pocket 200 is positioned substantially within the upper leg portion 107 of the leg region 106. A right releasable pocket 200 is depicted in a closed state in FIG. 1 and is depicted in an open state in FIG. 2. Moreover, as can be appreciated from FIG. 3 and additional discussion herein, the illustrated embodiment includes a releasable pocket 200 on the left leg. Other embodiments may include only one releasable pocket 200. In still other embodiments, the pants 100 may not include any releasable pockets.

For those embodiments that do include one or more releasable pockets 200, each pocket 200 may be configured to carry large and/or heavy items that are not conveniently carried in a pocket having a typical cloth construction. As can be appreciated, heavy and/or unconstrained objects contained in a typical pocket may move relative to the wearer's leg and hinder movement of the wearer, possibly quickly damaging the pants, and/or injuring the wearer. One or more of these disadvantages can be alleviated or resolved by the releasable pocket 200 and/or additional features discussed herein.

The releasable pocket 200 may have an outer pocket 211 with similar characteristics to a typical pocket. In some embodiments, the covering 101 of the pants 101, which may include an exterior portion of releasable pocket 200, may be made to appear similar to or substantially imperceptibly different from pants having a known construction, which are not configured for carrying large and/or heavy items. Accordingly, a casual observer may not readily notice or distinguish a difference between the tactical pants 100 and the pants having a known construction. In some embodiments, at least a portion of the pocket 200 may extend outwardly from adjacent portions of the pants 100.

With reference to FIG. 2, the releasable pocket 200 may be opened by decoupling a first closure 212, fastener, connector, or the like from a complementary second closure 213, fastener, connector, or the like. The closures 212, 213 may be constructed of hook and loop segments in some embodiments. In other embodiments, the closures 212, 213 may comprise one or more zipper elements, magnets, snaps, or the like. In the illustrated embodiment, the closures 212, 213 extend along at least two sides of the pocket 200, which is substantially rectangular in the illustrated embodiment. Accordingly, when the releasable pocket 200 is opened, such that a flap portion thereof can be expanded outwardly and/or folded downwardly, access to features inside the pocket is made considerably easier than can be achieved with a standard pocket having an opening along a single edge.

The releasable pocket 200 may include, or may be configured to house or enclose, a module assembly, utility assembly, or utility module 250, which is discussed further below. In some embodiments, the utility module 250 can include a holster 290 that is configured to receive a firearm.

With continued reference to FIGS. 1 and 2, in certain embodiments, the pants 100 can include one or more integrated tourniquet assemblies 500. In the illustrated embodiment, the pants 100 include an upper tourniquet assembly 500u and a lower tourniquet assembly 500l. The upper and lower tourniquet assemblies 500u, 500l can be said to be at different longitudinal positions relative to the leg portion 106 of the pants 100, as the leg portion 106 is elongated in a longitudinal direction. The specific positions of the tourniquet assemblies 500u, 500l can be strategically predetermined such that the assemblies can be used to constrict or close a critical blood vessel so as to reduce or stop blood flow to a region of a leg of a wearer of the pants 100. A wearer can select the region of the leg at which he or she wishes to constrict the blood flow, which may be accomplished quickly and without removing the pants 100, or in some instance, any portion of the pants 100 generally, or any portion of the leg portion 106. In some embodiments, selection of the area at which a tourniquet is to be applied can be facilitated by having multiple tourniquet assemblies within the leg portion 106. As further discussed below, in some embodiments, a lateral strap device, lateral strap assembly, or lateral strap system 240 (see FIGS. 2 and 3), which can typically be used for supporting a firearm or other utility device against a leg of the wearer, can also be used as a tourniquet assembly. Accordingly, the illustrated embodiment includes three tourniquet assemblies—the assemblies 500u, 500l and the lateral strap system 240—which are located at fixed positions relative to the leg portion 106, and which can be used to quickly provide a tourniquet to the leg of the wearer. Although the tourniquet assemblies are at fixed positions relative to the leg portion 106, it is appreciated that in some instances, the wearer may move the tourniquet assembly longitudinally (e.g., up or, in some embodiments, down) relative to his or her actual leg in order to locate the tourniquet assembly at the desired position on the leg.

As shown in FIGS. 1 and 2, the tourniquet assembly 500l is configured for rapid deployment in the event of the wearer suffering an injury. The assembly 500l includes a tourniquet member 501, which can comprise any suitable cord, strap, or other constricting device. In the illustrated embodiment, the tourniquet member 501 comprises a cord having a high strength capacity (e.g., type: 550 parachute cord). The tourniquet member 501 passes through a compartment 507 that is at least partially defined by the pants material 102. In the illustrated embodiment, an outer sidewall of the compartment 507 is defined by the pants material 102, and an inner sidewall of the compartment 507 is defined by an additional piece of material (not shown) that is attached to the pants material 102 via stitching 508. In the illustrated embodiment, two lines of stitching 508 are respectively positioned above and below the tourniquet member 501 and run substantially parallel to each other. In other embodiments, the pants material 102 may not directly define a portion of the compartment 507. For example, in some embodiments, a flexible tube or other compartment member may be attached to the pants material 102 in any suitable manner, and the tourniquet member 501 can extend through the tube. In various embodiments, the compartment 507 can extend about at least a majority of a perimeter of the leg portion 106 of the pants 100.

As shown in FIG. 2, an end of the tourniquet member 501 can extend through an opening 130 defined in the pants material 102. A stop 512 can be attached to the end of the tourniquet member 501, which can prevent the tourniquet member 501 from being withdrawn into the compartment 507. The stop 512 can also be conveniently graspable to aid in drawing portions of the tourniquet member 501 from the compartment 507 so as to tighten the tourniquet member 501.

In certain embodiments, one or more of the tourniquet assemblies 500u, 500l can each include a protective flap 509, which can cover the opening 130 and the stop 512. In some embodiments, one or more of the tourniquet assemblies 500u, 500l can include a torque bar or torque rod 511 that is used in tensioning the tourniquet member 501, as further discussed below. Any suitable number of torque bars 511 may be positioned within the pants 100, as desired. In the illustrated embodiment, each pocket 200 includes a small compartment 510 that retains a torque bar 511 therein. The pocket 200 or other regions of the pants 100 can include other or further compartments for housing other or further utility items.

FIGS. 3-5B and 13 illustrate an embodiment of a weight distribution system or harness 201 that can be integrated into the tactical pants 100. The harness 201 can include a longitudinal strap device, longitudinal strap assembly, or longitudinal strap system 220 and the lateral strap system 240 mentioned above. As discussed hereafter, the longitudinal strap system 220 can be configured to redistribute weight of a utility object in a substantially longitudinal direction (e.g., along a lengthwise direction of a leg of the pants), such that the weight is supported from the waist of a wearer of the pants 100 via the longitudinal strap system 220. The lateral strap system 240 can be configured to redistribute weight in a substantially lateral direction (i.e., in a direction that is transverse to the longitudinal direction). For example, the lateral strap system 240 can be configured to support weight against a leg of a wearer of the pants 100. In some embodiments, the harness 201 may include only one of the strap systems 220, 240.

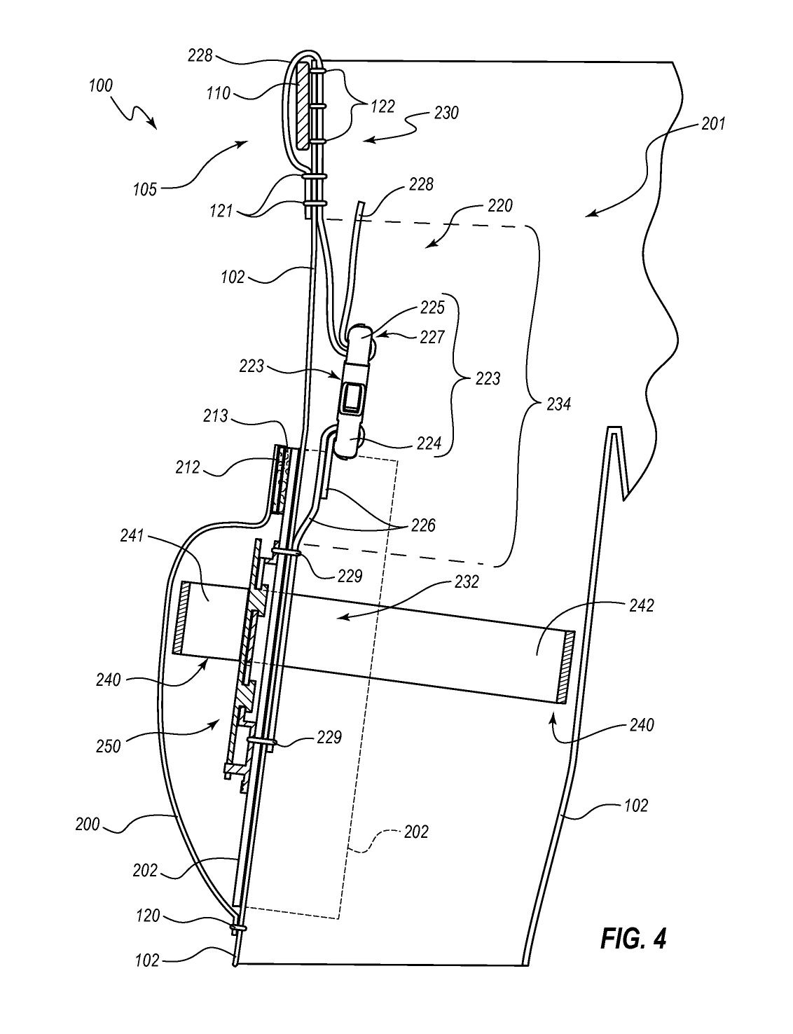

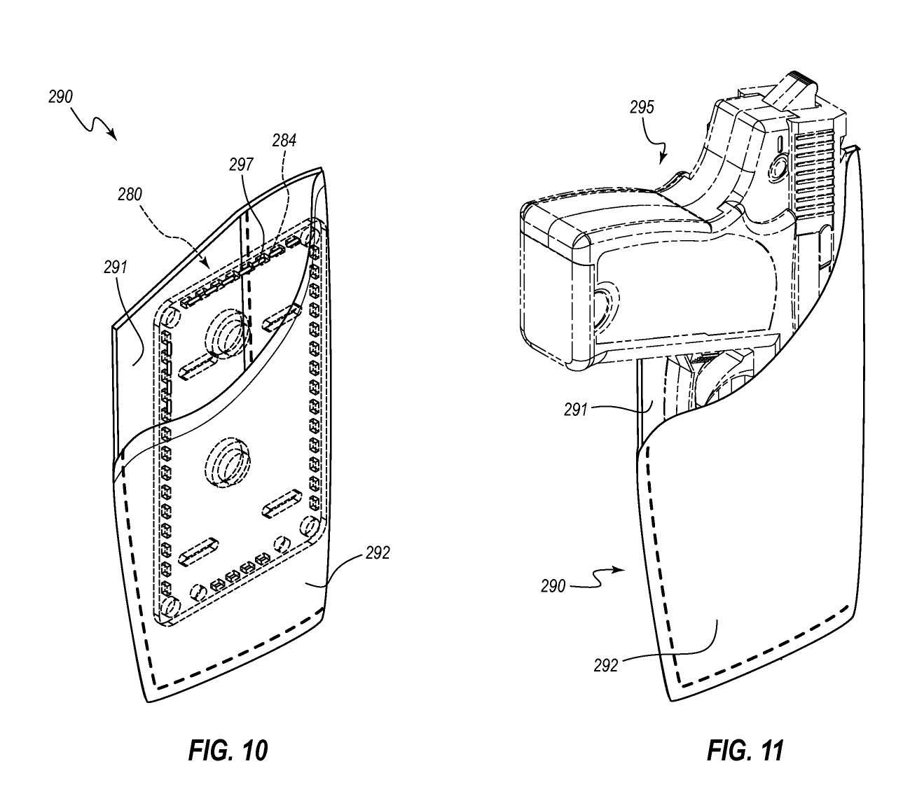

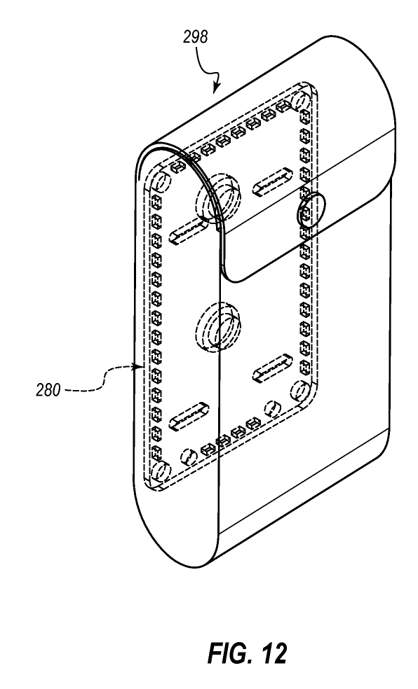

FIG. 3 shows the harness 201 with the pants material 102 hidden from view, but outlined in broken lines for reference. With reference to FIGS. 3 and 4, the harness 201 can distribute weight from an object contained in the releasable pocket 200 to the wearer by constraining the object to the wearer's leg using the lateral strap system 240, and can distribute weight vertically up to the waist region 105 of the pants 100 via the longitudinal strap system 220. The utility module 250 can be configured to retain a utility object, such as a firearm or other large and/or weighty item. For the sake of clarity, the utility module 250 is shown only in part in FIGS. 3 and 4. For example, the utility module is shown without a holster for retaining a firearm (such as the holster 290 discussed with respect to FIGS. 10 and 11), or without a utility pouch for retaining any other suitable item or items (such as that discussed with respect to FIG. 12).

As shown in FIG. 4, the utility module 250 can be at an interior of the releasable pocket 200. The utility module 250 can be attached to the pants material 102 in any suitable manner, such as via the stitches 229. In some embodiments, in order to enhance the comfort of the wearer, a pad or a liner 202 (see also FIG. 3) can be positioned at an inner surface of, or interiorly relative to, the utility module 250. The liner 202 can comprise any suitable material, and may provide a cushioning effect in some embodiments. The stitches 229 may pass through a portion of the utility module 250, the liner 202, and the pants material 102 in order to couple the utility module 250 to the pants material 102. As further discussed below, in the illustrated embodiment, the stitches 229 additionally pass through a portion of the longitudinal strap system 220 so as to couple the utility module 250 to the longitudinal strap system 220. Such a coupling is relatively direct, as the same stitching is used to tightly attach the components to each other. In other embodiments, the coupling of the utility module 250 and the strap system 220 may be more indirect. For example, the utility module 250 may be coupled to the pants material 102 via a first set of stitching or other fasteners, and the longitudinal strap system 220 may be coupled to the same or about the same area of the pants material 102 via a second set of stitching or other fasteners.

With reference to FIGS. 3-5B, the longitudinal strap system 220 can include an upper strap 228 and a lower strap 226. The upper and lower straps 228, 226 can be selectively coupled to each other and selectively decoupled from each other via a buckle 223 or any other suitable device. In the illustrated embodiment, the buckle 223 includes an upper connector 225 and a lower connector 224. Stated otherwise, the buckle 223 can allow the upper strap 228 to be connected to or disconnected from the lower strap 226, which is mechanically attached to utility module 250.

As previously discussed, the lower strap 226 can be attached to the pants material 102 via the stitches 229, or in any other suitable manner. The stitches 229 may mechanically couple the lower strap 226 to the utility module 250. The position at which the lower strap 226 is attached to the pants material 102 may be referred to as a lower anchor region 232.

In the illustrated embodiment, the lower strap 226 passes through the lower connector 224 and then is doubled back on and fixedly attached to itself. Accordingly, the lower strap 226 does not provide any adjustability to a length of the longitudinal strap system 220.

The upper strap 228 can be attached to the pants material 102 via any suitable fastening system. In the illustrated embodiment, the upper strap 228 is attached to the waist portion 105 of the pants 100 via a first set of stitches 121 and a second set of stitches 122. The upper strap 228 is looped over an upper end of the pants material 102 so as to pass from an interior of the pants material 102 to an exterior thereof. The exterior end of the upper strap 228 is attached to an interior portion of the upper strap 228 via the stitches 121. This arrangement forms a belt loop 204, which can receive the belt 110 so as to transfer weight to the belt 110. The belt loop 204 is also shown in FIGS. 1 and 2. The second set of stitches 122 can attach an interior portion of the upper strap 228 to an upper end of the pants material 102, and can provide a further connection between the upper strap 228 and the waist region 105. The position at which the upper strap 228 is attached to the pants material 102 may be referred to as an upper anchor region 230.

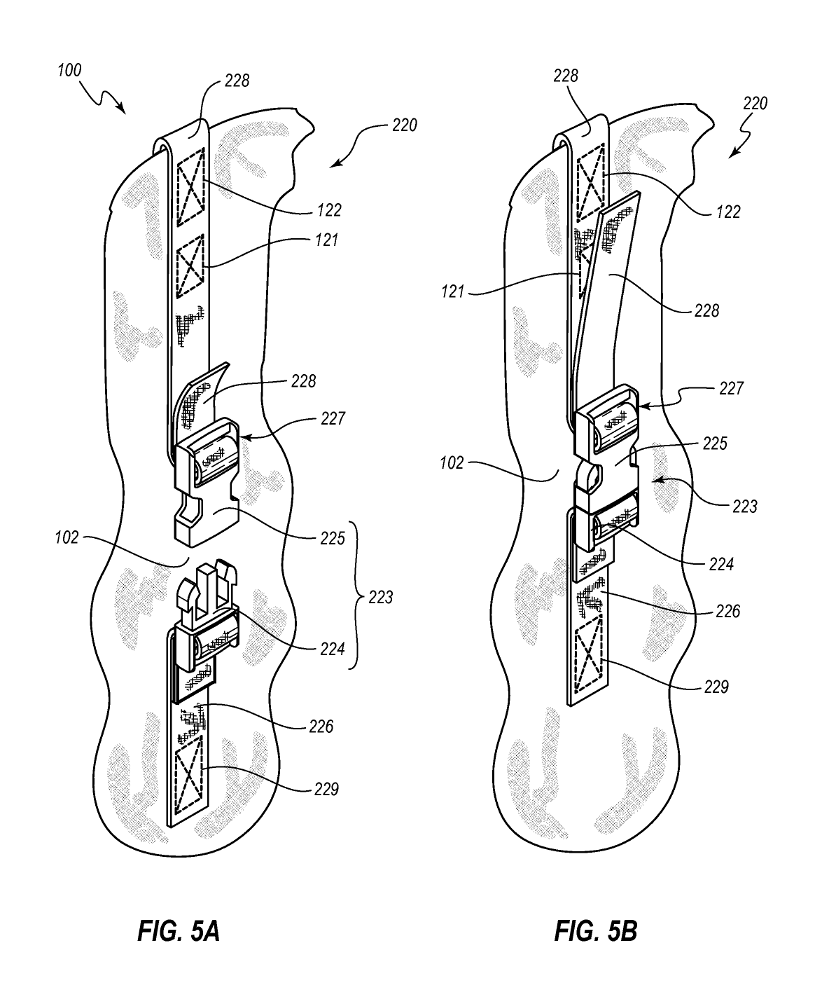

In the illustrated embodiment, the upper strap 228 passes through a tensioning portion 227 of the upper connector 225. A proximal end of the upper strap 228 is free and is graspable by a wearer of the pants 100. As shown in FIGS. 5A and 5B, the upper strap 228 can be advanced through the tensioning portion 227 of the upper connector 225 in opposite directions so as to adjust the position of the upper connector 225 relative to the upper end of the pants 100. The tensioning portion 227 of the upper connector 225 can maintain the upper connector 225 at the position to which it has been moved. The upper connector 225 may also be referred to as a tensioning member.

Movement of the upper connector 225 in the manner just described can alter a length of the longitudinal strap system 220. For example, as shown in FIG. 5A, a relatively small proximal portion of the upper strap 228 may be available for grasping, such that the longitudinal strap system 220 is relatively long when the connectors 224, 225 are coupled with each other. With the connectors 224, 225 coupled, the free end of the strap 228 can be pulled so as to advance the upper connector 225 upwardly to the position shown in FIG. 5B. This can effectively reduce the length of the longitudinal strap system 220, and can increase the tension in the straps 226, 228. A wearer of the pants 100 thus can adjust a length of the longitudinal strap system 220, as desired. The adjustment may alter an amount of the weight of the utility module 250, and the weight of any utility object retained thereby, that that the waist portion 105 and/or the belt 110 bears. The longitudinal strap system 220 can be used to redistribute at least a portion of the weight of the utility module 250, and any utility object retained thereby, from the pants material 102 to the straps 226, 228.

The portion of the longitudinal strap system 220 that is not connected to the pants material 102 may be referred to as an unconnected segment 234 of the longitudinal strap system 220. The unconnected segment 234 can include the portions of the upper and lower straps 228, 226 that extend between the upper and lower anchor regions 230, 232. The unconnected segment 234 is separate from the pants material 102, and thus, in many instances, a length of the unconnected segment 234 can be adjusted without affecting a length of any neighboring portion of the pants material 102. This may be particularly so where the unconnected segment 234 is slack, and the length of the unconnected segment 234 is adjusted to reduce or eliminate the slack.

As previously discussed, the strap 228 may form the belt loop 204, which can be supported by the belt 110, as shown in FIGS. 3 and 4. Any suitable belt 110 is possible. With reference to FIG. 3, in the illustrated embodiment, the belt 110 includes a pair of complementary connectors 111, 112 that interconnect as a buckle. A portion of the belt 110 can be pulled through the buckle so as to tighten the belt 110 to the torso of the wearer, and the belt can thereby support the strap 228.

In various embodiments, one or more strength properties of the straps 226, 228 are greater than those of the pants material 102. For example, a fabric material used in the straps 226, 228 can be configured to stretch less than the pants material 102 would under an equivalent load. In some embodiments, the straps 226, 228 comprise nylon webbing.

With reference to FIG. 4, a portion of the releasable pocket 200 may be attached to the pants material 102 via stitches 120, or by any other suitable fastening system. As previously discussed, other portions of the releasable pocket 200 (e.g., a top edge and at least a portion of a side edge), can be coupled to the pants cloth via the closures 212 and 213.

With reference to FIGS. 3 and 4, a portion of the lateral strap system 240 can be positioned within the releasable pocket 200 and can encompass or extend about the utility module 250, as discussed further below. The lateral harness 240 can extend through the liner 202 and the pants material 102 into an interior of the leg portion of the pants 100. The portion of the lateral strap system 240 that is at an interior of the pants 100 can be independent of or unattached to the pants material 102. A wearer of the pants can insert a leg through the lateral strap system 240, and may adjust a length of the lateral strap system 240 as desired. In the illustrated embodiment, adjustment of the length of the lateral strap system 240 can occur independently of, or without affecting, the shape or contour of the pants material 102. Stated otherwise, a perimeter defined by the lateral strap system 240 can be altered without altering a perimeter defined by a neighboring portion of the pant leg. Such an arrangement can allow for adjustment of the lateral strap system 240 without bunching the pants material 102. This can provide comfort to the wearer and may avoid visual indications that the wearer is carrying a large or heavy object in the pants 100. However, in other embodiments, the lateral strap system 240 may not be operative independently of the pants material 102. For example, in some embodiments, the pants material 102 may define at least a portion of a compartment through which the lateral strap system 240 is threaded, which may resemble the tourniquet assemblies 500 discussed above.

As previously discussed, the utility module 250 may be used to hold a variety of devices, depending on the preferences of the wearer. The utility module 250 may be configured to quickly or readily interchange the type of device that is held within the pocket 200.

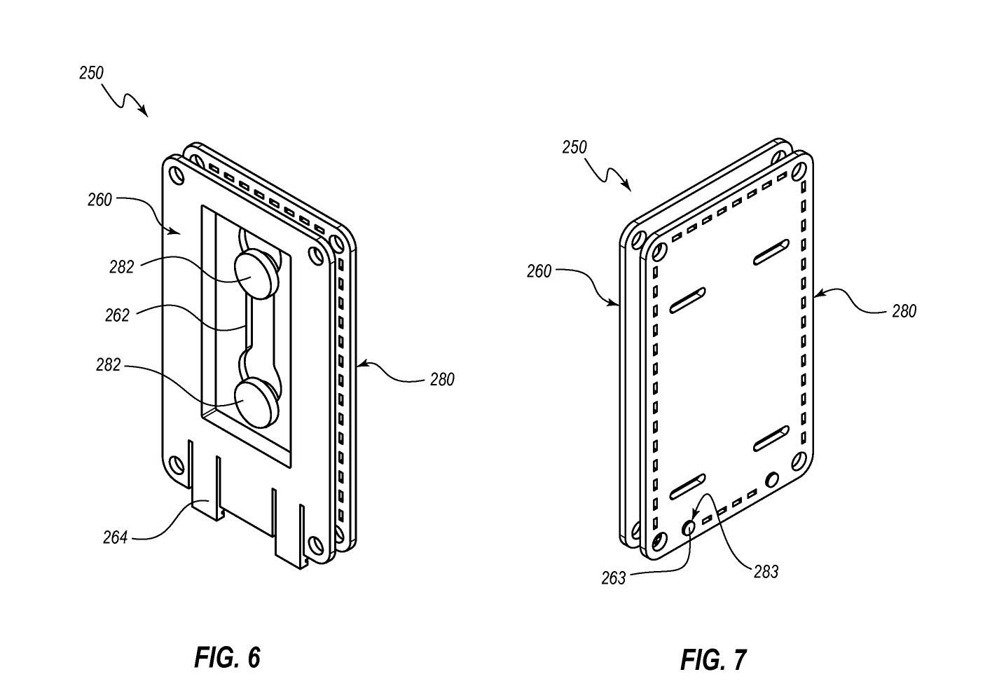

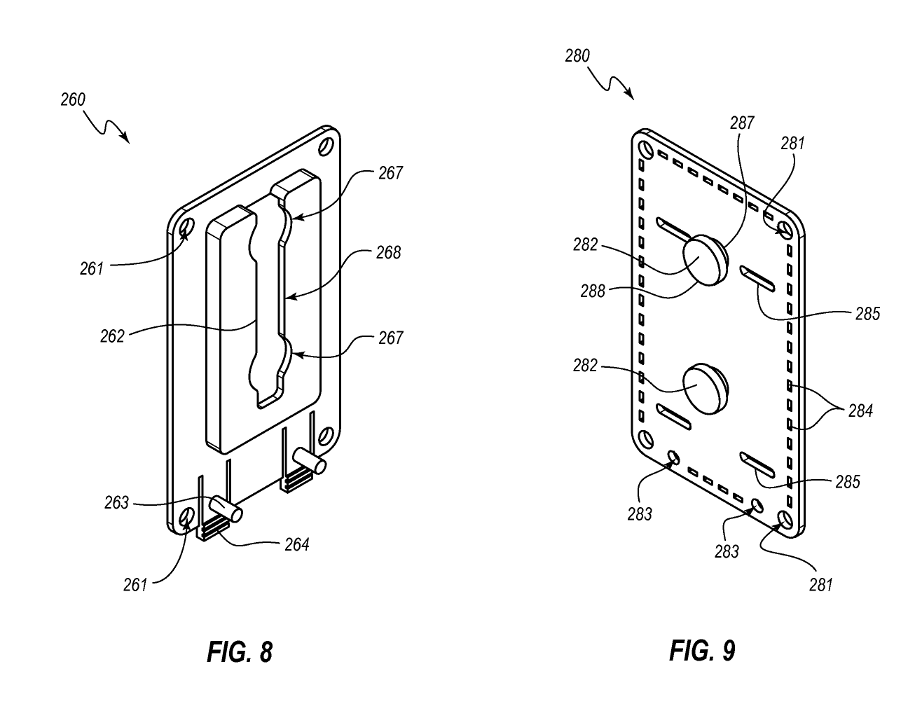

FIGS. 6-9 illustrate a module assembly 250 for carrying utility items that can be quickly attached and detached from the tactical pants 100. FIG. 6 depicts a base, or an inner bracket 260, and a mount, or an outer bracket 280, that are connected together. The inner bracket 260 can be mechanically attached to the liner 202 and/or pants material 102 of the tactical pants 100 in any suitable manner. In the illustrated embodiment, the inner bracket 260 includes through holes 261 through which any suitable fastener can be advanced, such as the stitching 229 discussed above, rivets (not shown), or any other suitable devices. The inner bracket 260 can define a slot 262. The slot 262 may include two widened regions 267 that extend outwardly relative to a narrow region 268.

The outer bracket 280 can include protrusions 282, which may include knob or disk-like extensions 288 that extend laterally outward from a narrow post 287. The extensions 288 can be sized to fit into the widened regions 267 of the slot 262, yet can be too large to be removed from the narrow region 268 of the slot 262. Accordingly, the outer bracket 280 can be coupled to the inner bracket 260 by introducing the extensions 288 into the widened regions 267 of the slot 262. The outer bracket 280 can be slid downward so as to advance the extensions 288 into proximity with the narrow region 268 of the slot 262, which can retain the outer and inner brackets 280, 260 in a coupled configuration.

In certain embodiments, the brackets 260, 280 may include a system for further assisting in maintaining the coupled configuration. In the illustrated embodiment, quick release pins 263 of the inner bracket 260 can be introduced into the holes 283 of bracket 280 to lock the assembly together. Resilient tabs 264 can be depressed so as to translate the pins 263 out of the holes 283 to release bracket 280 from the bracket 260.

The outer bracket 280 can include through holes 284 and/or slots 285 for coupling a utility object retaining device thereto. Any suitable fastening mechanisms may be used to attach the retaining device to the outer bracket 280. For example, stitching, adhesives, screws, magnets, and/or rivets may be used. Two examples of retaining devices that may be coupled with an outer bracket 280 are provided in FIGS. 10-12, as discussed hereafter.

FIG. 10 illustrates a holster 290 coupled with the outer bracket 280. The holster 290 may have a holster back 291 and a holster front 292. The holster 290 is attached to the outer bracket 280 by stitching 297 through the holster back 291 and stitching holes 284. FIG. 11 illustrates the utility of the holster module 290 with a firearm 295 positioned in the pocket formed between the holster back 291 and holster front 292.

FIG. 12 illustrates a utility pouch 298 that can be used to retain any suitable utility item. The utility pouch 298 can be mounted to the bracket 280 in any suitable manner.

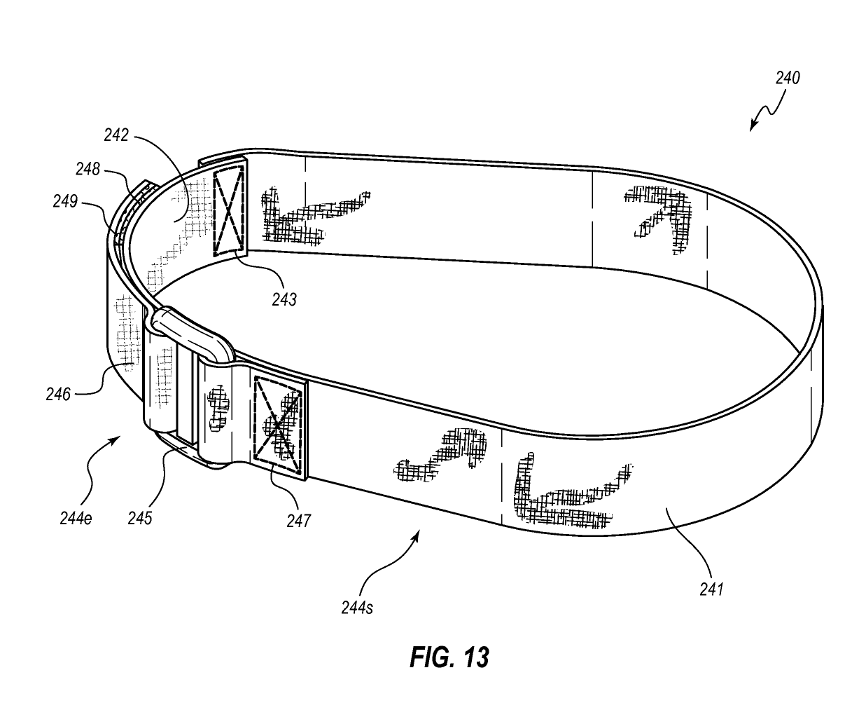

As can be appreciated from the foregoing, the module assembly 250 can be used to retain a utility object within the pocket. The lateral strap system 240 may be used to support the module assembly 250, and any utility object retained thereby, against a leg of a wearer of the pants 100. Examples of lateral strap systems 240 are provided in FIGS. 13-15, as discussed immediately hereafter. With reference again to FIGS. 3 and 4, the lateral strap system 240 can pass through the liner 202 and the pants material 102 at two separate locations. As shown in FIG. 2, a portion of the lateral strap system 240 can be exposed when the releasable pocket 200 is in the open orientation. In various embodiments, the exposed portion can extend, for example, about the holster 290 or the utility pouch 298, depending on the construction of the module assembly 250. In some embodiments, the exposed portion can be used to adjust a length and/or adjust a tension of the lateral strap system 240.

FIG. 13 illustrates an embodiment of the lateral strap system 240 that is compatible with the harness 201 of FIGS. 3 and 4. A length of or perimeter defined by the lateral strap system 240 can be adjusted, which can adjust a tension of the strap system 240 about the leg of a wearer. In various embodiments, the lateral strap system 240 can be positioned so as to support the module assembly 250 against a thigh of the wearer. Any suitable mechanisms may be used to selectively adjust the lateral strap system 240. In the illustrated embodiment, the strap system 240 includes a loop 245 and a strap 242 that is advanced through the loop 245 and doubled back on itself. The strap 242 includes complementary attachment features or connectors 248, 249 that face each other so as to couple with each other. In the illustrated embodiment, the connectors 248, 249 comprise complementary strips of hook and loop fastener material. Other suitable connectors 248, 249 are also possible, such as snaps, clamps, belt holes, catches, etc. A proximal end 246 of the strap 242 can be pulled so as to place the strap system 240 under tension around the leg of the wearer. In some embodiments, the proximal end 246 of the strap 242 is accessible to a wearer of the pants 100 via the pocket 200 (see FIG. 2).

In some embodiments, the strap system 240 includes a first strap 241 formed of a first material, and the strap 242 is formed of a second material. The first and second materials can have different elastic and/or extensibility properties. In some embodiments, the material of the first strap 241 can comprise a material that does not significantly stretch along its length, such as, for example, nylon webbing. The second strap 242 can comprise, for example, an elastic or resiliently deformable material. The first material of the first strap 241 thus may be less extensible than the second material of the second strap 241. The elastic material of the second strap 242 can be configured to maintain a substantially constant tension in the lateral strap system 240 as small changes arise in the overall length of the strap system 240. The elastic material thus can permit the strap system 240, which can be tensioned about a leg of a wearer, to maintain a relatively constant tension, even as a perimeter length of the lateral strap system 240 changes to conform to changes in the perimeter of the leg due to, for example, muscle activity. In some embodiments, a 1 inch change in the length of the relatively flexible or elastic second strap 242 can result in a change in the tension of the lateral strap system 240 that is within a range of from about 1 to about 20 pounds, from about 1 to about 10 pounds, or from about 1 to about 5 pounds, or that is no less than 1, 2, 3, 4, 5, 10, or 20 pounds, or that is no greater than about 1, 2, 3, 4, 5, 10, or 20 pounds. The tension in the strap system 240 can be adjusted by decoupling the connector 249 from the connector 248, repositioning the proximal end 246 of the strap 242 relative to a more distal region thereof, and re-coupling the connectors 248, 249.

In view of the foregoing, a portion of the lateral strap system 240 may be defined as a support region 244s, and another portion of the lateral strap system 240 may be defined as an expansion region 244e. The support region 244s can generally contact the leg of a wearer and maintain a tension therewith so as to support the utility module against the leg. The expansion region 244e can resiliently expand and contract so as to maintain the tension at a relatively constant level, or stated otherwise, so as to keep the tension within a narrow range. The expansion region 244e may be particularly helpful when the lateral strap system 240 operates in a supporting mode, such as just described.

The expansion region may function in a different manner, or may not be used at all, when the lateral strap system 240 is used in a tourniquet mode. That is, in some embodiments, the lateral strap system 240 may be used as a tourniquet. For example, in various embodiments, one or more of the utility object that is retained by the utility module 250 and/or the bracket 280 and any items attached thereto may be removed from the pocket 200. The proximal end 246 of the strap 242 may be pulled so as to tighten the lateral strap assembly 240 and reduce blood flow to the lower leg. The strap 241 and/or the strap 242, whether together or individually, can act as a tourniquet member.

In some embodiments, the resiliently flexible strap 242 is pulled taut so as to substantially reach a stretching limit. The straps 241, 242 may thereafter maintain a substantially constant tension to reduce blood flow to the lower portions of the leg. In other embodiments, the resiliently flexible strap 242 may be pulled out of the way, and the relatively inelastic strap 241 may be advanced through loop 245. Accordingly, only the tension that is in the strap 241 may be used to reduce the flow of blood to the lower portions of the leg. In either case, additional fastener portions 248, 249 may be positioned on one or more of the straps 241, 242 so as to permit a greater portion of the lateral strap system 240 to be pulled through the loop 245 and fastened in place to maintain the higher tension thus achieved.

In some embodiments each of the straps 241 and 242 comprises an elastic material. In other embodiments, each of the straps 241, 242 comprises a relatively inextensible material. In still further embodiments, the lateral strap system 240 may include a unitary strap, which does not include separate expansion and support regions 244e, 244s.

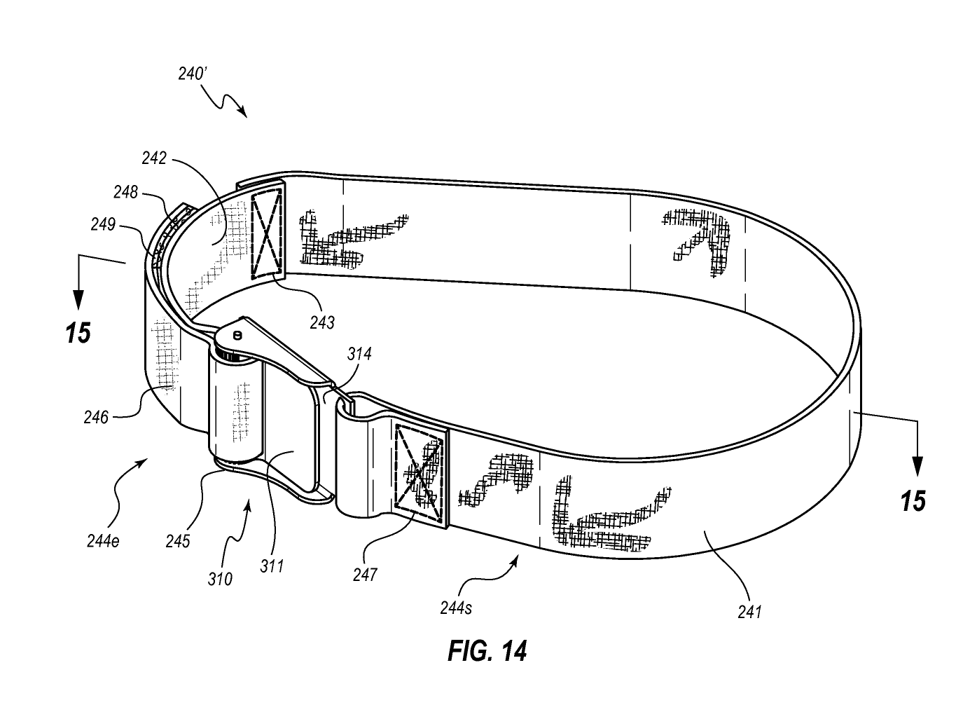

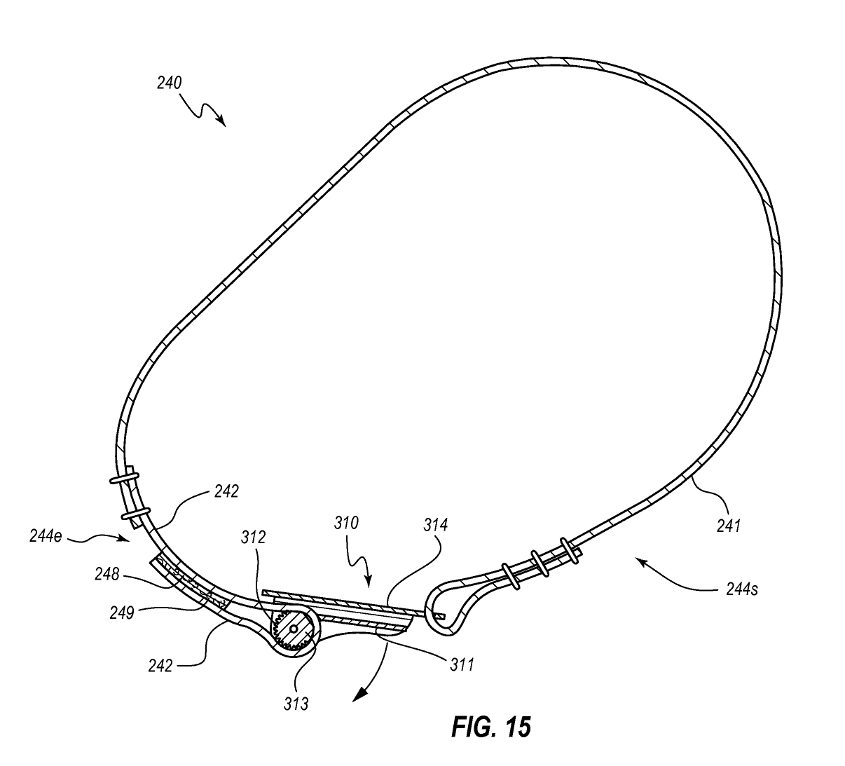

FIGS. 14-15 illustrate another embodiment of a lateral strap system 240′ that is compatible with the harness 201. The lateral strap system 240′ can include a tensioning locking assembly 310 that can be used to obtain a higher tension that that which may be achieved merely by manually gripping the distal end portion 246 of the strap 242. The locking assembly 310 includes a rotatable column 313 having an outer perimeter. A portion of the outer perimeter is smooth, while another portion thereof includes grips 312. The rotatable column 313 is selectively rotatable via a lever 311 that is connected thereto. The locking assembly 310 further includes a bracket 314.

In operation, the locking assembly 310 permits the strap 242 to freely travel between the smooth side of the column 313 and the bracket 314. Once a high tension has been manually achieved, the lever 311 can be rotated away from the bracket 314, which causes the grips 312 to engage the strap 242 and advance it through the locking assembly 310 even further, thus increasing the tension in the strap system 240′. The lever 311 may any suitable components, such as, for example, springs or detents (not shown) to lock the displaced lever 311 in place and thus maintain strap tension. The locking assembly 310 may include other and/or additional components to prevent the lever from being inadvertently or prematurely rotated from the initial state. These various components can include, for example, one or more clasps, ratchets, tabs, detents, fasteners, and hooks. The locking assembly 310 thus can assist a wearer to achieve a high, substantially fixed tension, due to the mechanical advantage of the lever and its locking capabilities. Other suitable arrangements are also possible for the locking assembly 310, such as, for example, standard ratcheting systems.

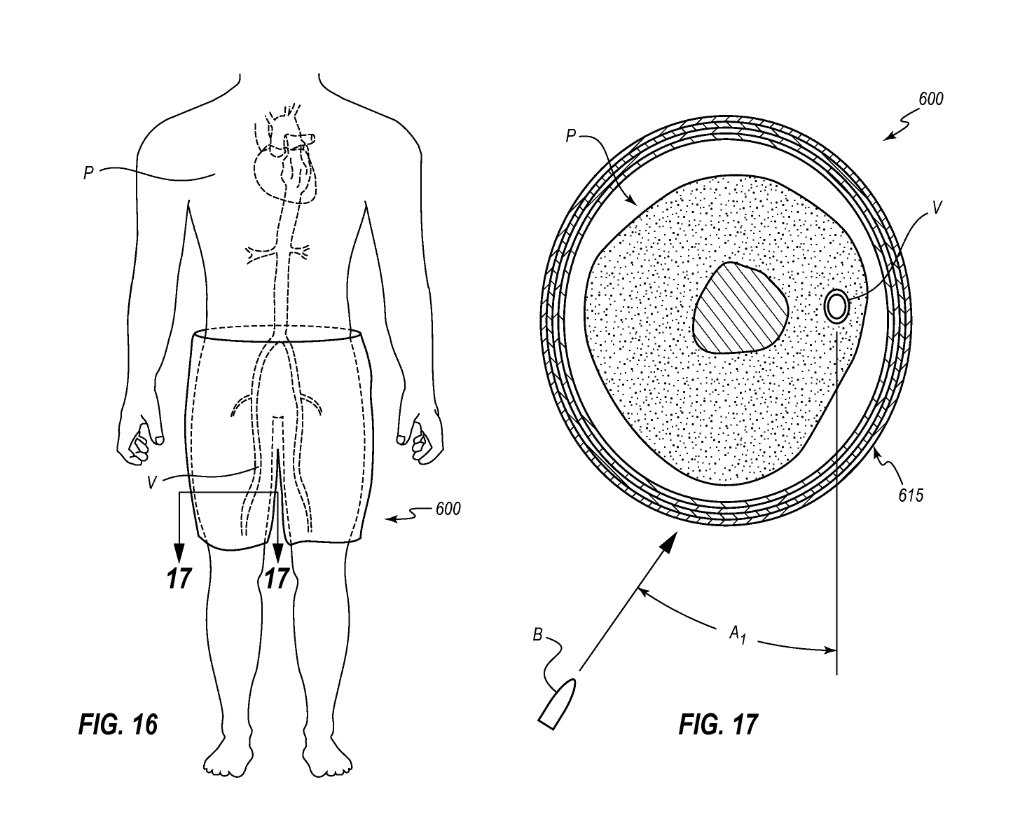

FIGS. 16 and 17 illustrate a person P wearing protective shorts, body armor, or ballistic-resistant shield 600 around his lower torso and upper leg region. The ballistic-resistant shield 600 provides protection around critical blood vessels from one or more ballistic projectiles B traveling toward the person P. An example of a critical blood vessel V is the femoral artery, as injury to this artery may quickly cause significant injury or death. In some embodiments, the ballistic-resistant shield 600 creates a sleeve that fully encompasses the critical blood vessel V, providing 360-degree protection (along a horizontal plane) when the person P is upright. Stated otherwise, an arbitrary or predetermined horizontal line in a horizontal plane may extend through the critical blood vessel V when the person P is upright. The ballistic-resistant shield 600 may protect the critical blood vessel V from ballistic projectiles B that approach the critical blood vessel V along a trajectory that is within an angle A₁ relative to the horizontal line. In the illustrated embodiment, the value of the angle A₁ over which the ballistic-resistant shield 600 will provide protection to the vessel V is 360 degrees. As can be seen in FIG. 16, the ballistic resistant shield 600 can fully encompass a femoral artery along at least a portion of the length of the femoral artery.

While the full 360 degree protection can be desirable, some embodiments of the ballistic-resistant shield 600 can limit mobility of the person P. Accordingly, in some embodiments, mobility-increasing features are employed with the ballistic-resistant shield 600. For example, as further discussed below, in some embodiments, the ballistic-resistant shield 600 extends around only a strategically determined segment of the critical blood vessel V. In other or further embodiments, the ballistic-resistant shield 600 can include one or more mobility seams that provide increased mobility without sacrificing protection, or without significantly increasing vulnerabilities.

The protective garment 600 is constructed of ballistic-resistant armor 615 that can comprise any suitable ballistic-resistant material. For example, in various embodiments, the ballistic-resistant material comprises para-aramid synthetic material (e.g., Kevlar®), which may also be referred to herein as an aramid material. The term aramid fiber, as used herein, may describe the fiber itself, the woven fiber, or a composite of layers of woven fiber. In some embodiments, the ballistic-resistant armor comprises a plurality of aramid synthetic woven fiber layers that are attached together as a composite to increase the level of ballistic protection. In some embodiments, the ballistic resistant armor 615 may be a composite of aramid woven fiber layers sandwiched between the pants material and a protective or comfort-providing liner or layer, such as, for example, faux-suede liner.

As is commonly known, the National Institute of Justice (NIJ) has developed standards that define the level of threat protection by a protective garment. In various embodiments, the protective garment 600 provides protection from threat level IIA or greater, threat level II or greater, threat level IIIA or greater, or threat level III or greater under NIJ Standard 0101.06, July 2008. Threat level IIA is tested with 9 mm Full Metal Jacketed Round Nose (FMJ RN) bullets with a specified mass of 8.0 grams (124 grains) and a velocity of 373 m/s±9.1 m/s (1225 ft/s±30 ft/s) and with .40 S&W Full Metal Jacketed (FMJ) bullets with a specified mass of 11.7 g (180 gr) and a velocity of 352 m/s±9.1 m/s (1155 ft/s±30 ft/s). Threat level II is tested with 9 mm FMJ RN bullets with a specified mass of 8.0 g (124 gr) and a velocity of 398 m/s±9.1 m/s (1305 ft/s±30 ft/s) and with .357 Magnum Jacketed Soft Point (JSP) bullets with a specified mass of 10.2 g (158 gr) and a velocity of 436 m/s±9.1 m/s (1430 ft/s±30 ft/s). Threat level IIIA is tested with .357 SIG FMJ Flat Nose (FN) bullets with a specified mass of 8.1 g (125 gr) and a velocity of 448 m/s±9.1 m/s (1470 ft/s±30 ft/s) and with .44 Magnum Semi Jacketed Hollow Point (SJHP) bullets with a specified mass of 15.6 g (240 gr) and a velocity of 436 m/s±9.1 m/s (1430 ft/s±30 ft/s). Threat level III is tested with 7.62 mm FMJ, steel jacketed bullets (U.S. Military designation M80) with a specified mass of 9.6 g (147 gr) and a velocity of 847 m/s±9.1 m/s (2780 ft/s±30 ft/s).

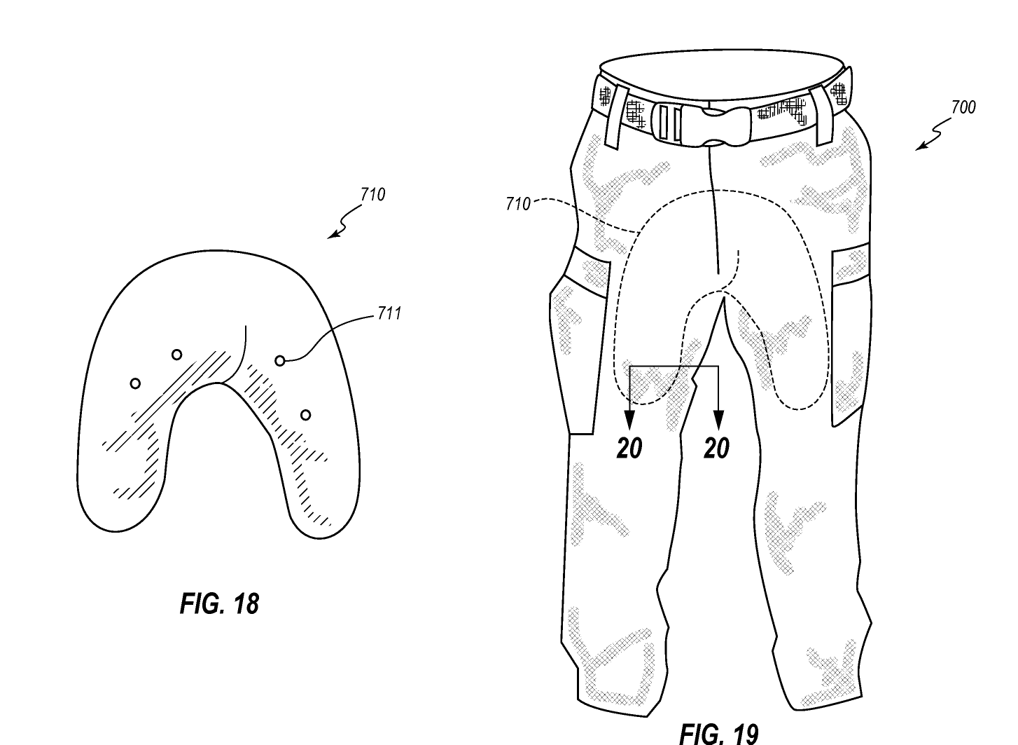

FIGS. 18-20 depict another embodiment of a pair of tactical pants 700 having a ballistic-resistant shield 710, which may also be referred to as a flak saddle 710, sewn into the crotch region of the pants. The tactical pants 700 can resemble the pants 100 described above, and in some embodiments, may vary from the pants 100 only with respect to the inclusion of a flak saddle 710 therein. The flak saddle 711 may be stitched or otherwise attached to an interior of the pants 700.

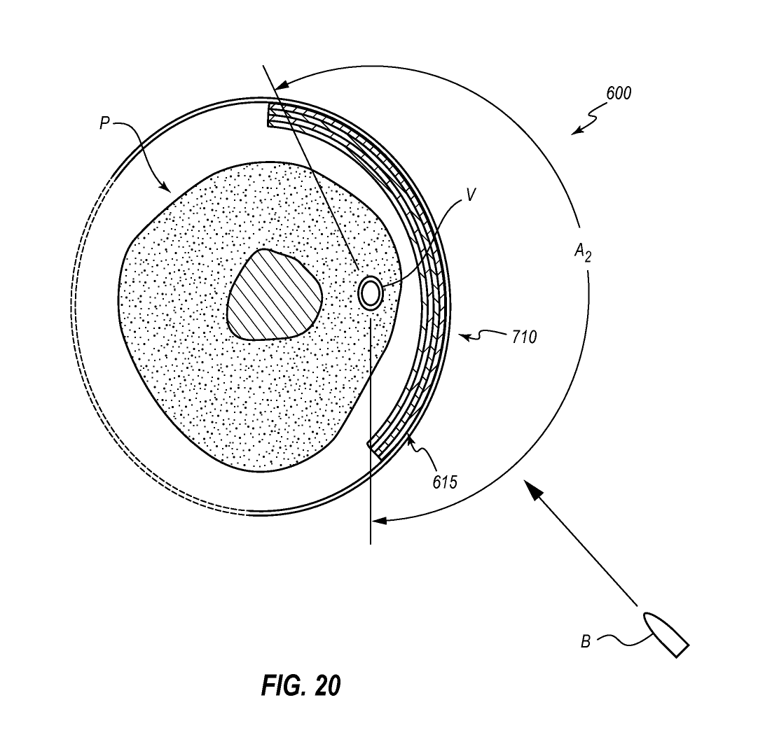

The flak saddle 710 may have vents 711 in the ballistic material that are small so to prevent penetration by a projectile B, but are large enough to allow air to flow through the material to keep the wearer comfortable. As shown in FIG. 20, the flak saddle 710 may provide only a strategic segment of protection around the critical blood vessel V. This may provide protection for a significant range of threats from projectiles directed toward the critical blood vessel V, while increasing mobility of the person P, as compared to certain full-sleeve protection garments. In some embodiments, the flak saddle 710 provides a range A₂ of protection around the critical blood vessel V that is no less than about 25, 35, 45, 50, 60, 75, 80, 90, 100, 130, 140, 150, 160, 170, or 180 degrees.

As shown in FIG. 20, when the flak saddle 710 is incorporated into the pants 700, a leg portion of the saddle 710 can cover at least a portion of an internal perimeter of the leg region of the pants 700. This internal coverage can extend longitudinally along an upper portion of the leg region of the pants 700 (see FIG. 19). In various embodiments, the leg portion of the saddle 710 covers no less than one quarter, one third, or one half of the internal perimeter of the leg region of the pants 700.

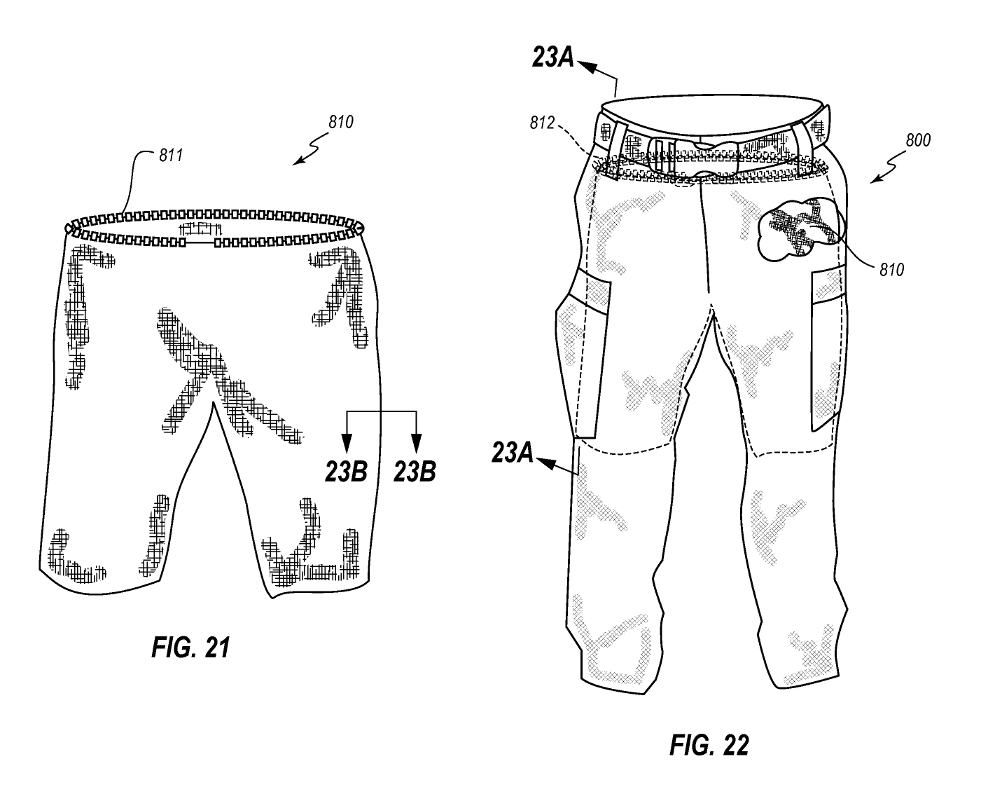

FIGS. 21-22 depict another embodiment of a pair of tactical pants 800 that include a ballistic-resistant shield 810, which can be attached to the pants by fasteners 811 and 812. In some embodiments, the fasteners may be a zipper or hook-and-loop material segments. In other or further embodiments the ballistic-resistant shield 810 may be sewn into the tactical pants 800. Additional material layers (e.g., a liner) may also be sewn into the pants 800 at an interior of the ballistic-resistant shield 810 to provide comfort to the wearer.

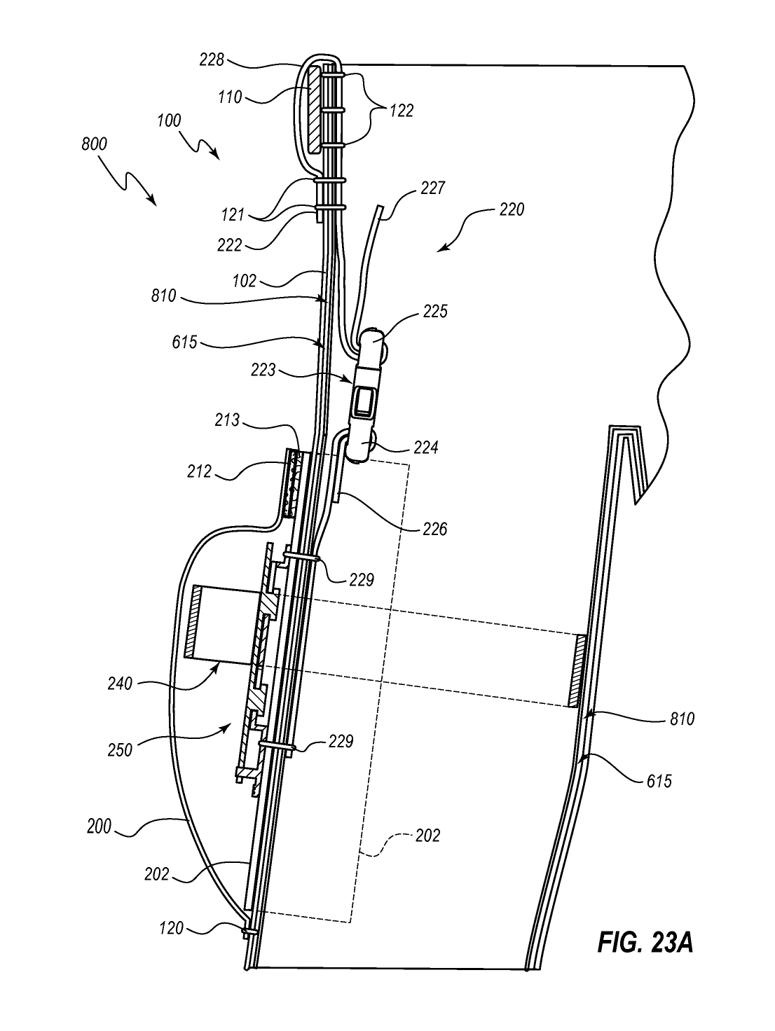

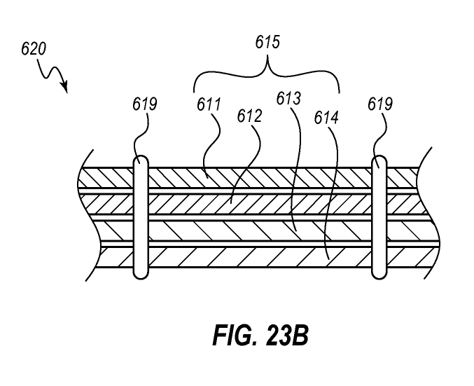

FIG. 23A is a cross-sectional view of the pants 800 of FIG. 22, which shows that a portion of the lateral strap system 240 extends through the ballistic-resistant shield 810 into an interior of a leg portion of the pants 800. In further embodiments, the outer pocket 200 may have anti-ballistic material attached at an interior side thereof. In certain of such embodiments, the lateral strap system 240 can feed through the liner 202 into the pocket 200. A wearer of the pants 800 can be less vulnerable to ballistic projectiles passing through the openings via which the lateral strap system 240 feeds into the pocket 200 due to the presence of the anti-ballistic material in the pocket.

FIG. 23B is a cross-sectional view of a flexible four-layer composite construction, or composite material 620. By using a composite material 620 that is composed of thin layers of ballistic-resistant cloth, the garment construction can have increased flexibility to provide fewer impediments to the wearer's movement. To achieve increased level of ballistic protection, the layers 611, 612, 613, and 614 are intermittently bound together by stitching 619 to form a ballistic-resistant armor 615. One or more of the layers 611, 612, 613, and 614 can comprise a woven aramid fiber.

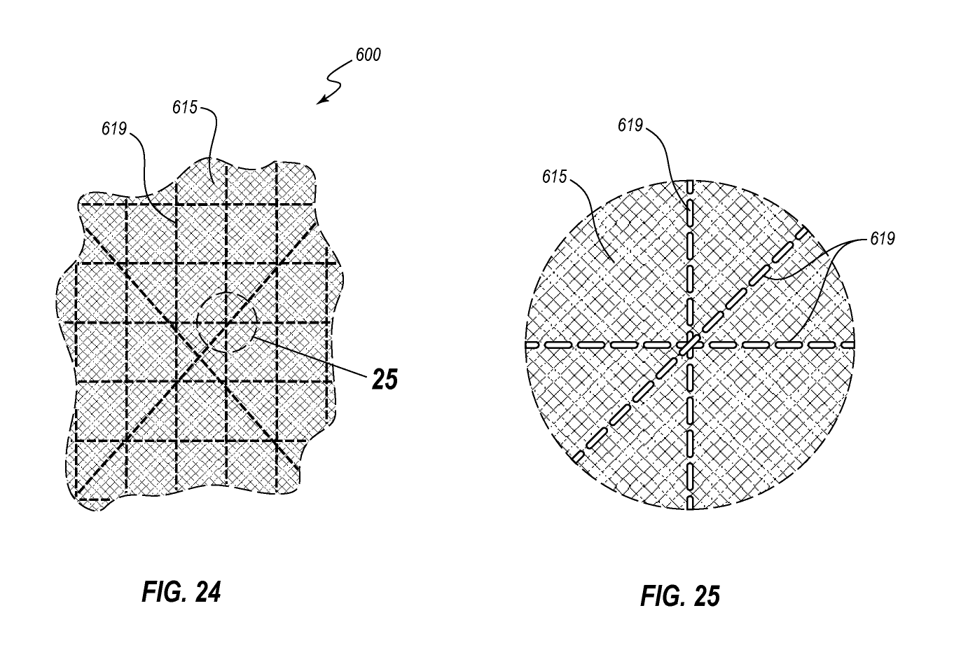

FIGS. 24 and 25 depict portions of the ballistic resistant armor 615 used in construction of the ballistic-resistant shield 810. Stitching 619 in various orientations is used to bind the woven fiber layers (e.g., 611, 612, 613, 614) together to achieve higher levels of ballistic protection. In some embodiments, the stitching 619 orientations and spacing can be optimized to provide a high degree of ballistic protection while allowing flexibility for movement of the wearer. An adhesive (not shown) may also be used to cause the aramid fiber layers to work together as a composite structure for increased protection. In various embodiments, spacing between adjacent stitches may be no greater than about 0.5, 1.0, 1.5, or 2.0 inches.

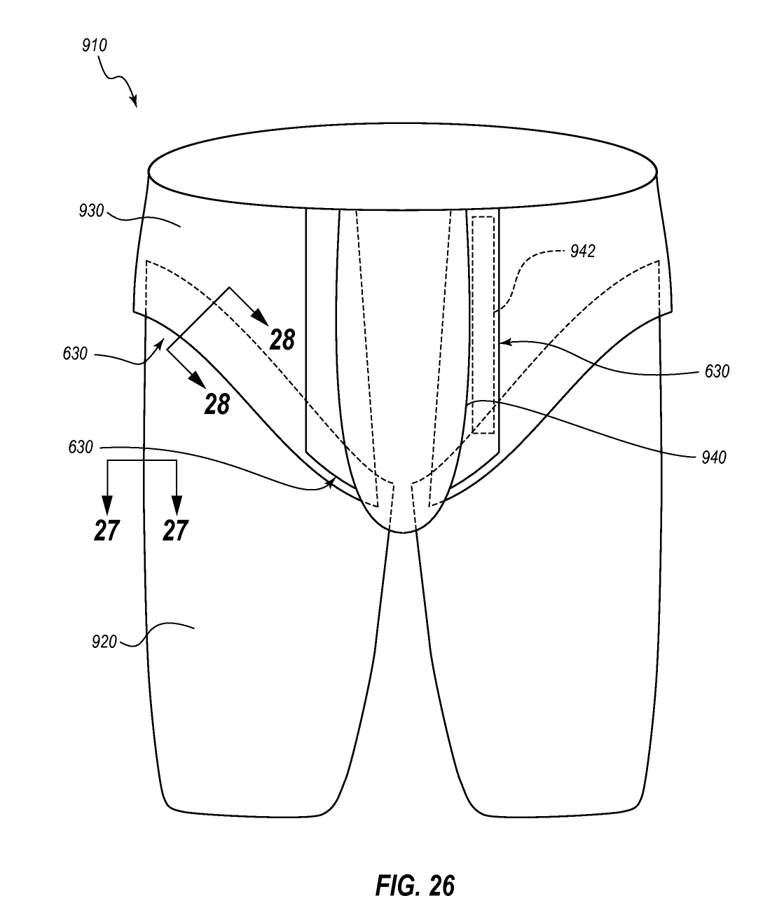

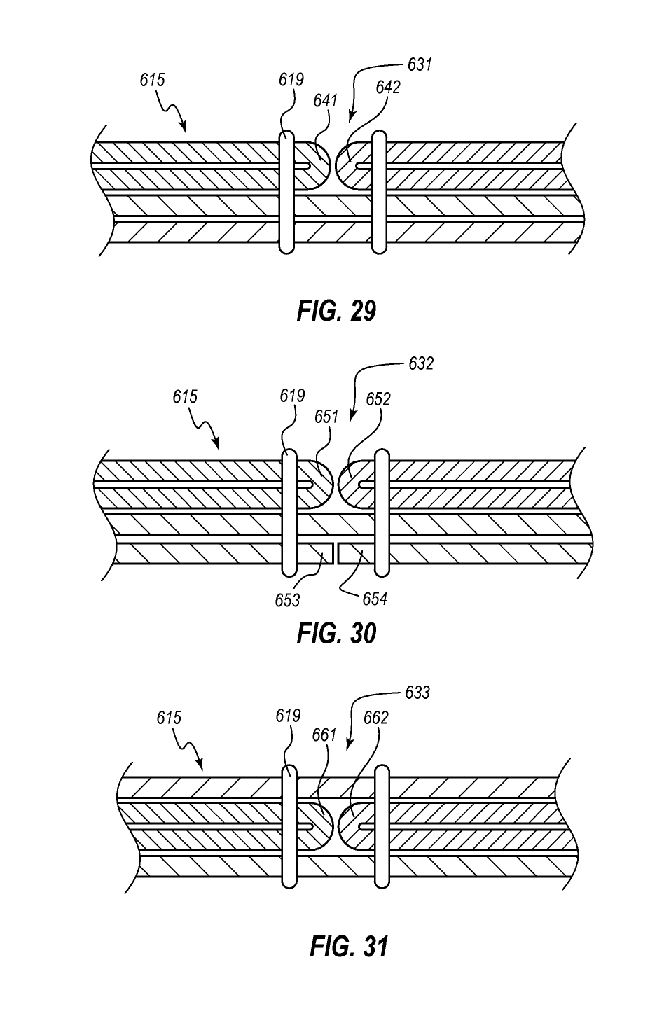

FIG. 26 illustrates another embodiment of a ballistic-resistant shield 910 that can be integrated into any of the tactical pants described herein. The ballistic-resistant shield 910 can include a leg section 820, a waist or pelvic section 830, and a groin section 840. The various sections 820, 830, 840 are overlapping so as to provide flexible or mobility seams 630 at the anatomical joints of the wearer. The illustrated seam 630 is discussed below with respect to FIG. 28. Other suitable arrangements of seams are discussed with respect to FIGS. 29-34. One or more of the seam configurations can be used in any suitable combination with any suitable ballistic-resistant shield 910. The seams can increase mobility of the wearer, as bending or other movement can occur more readily at the seams than at other portions of the composite materials. In some embodiments, the ballistic resistant shield 910 can include an access flap 942, which in further embodiments, may be selectively sealed closed.

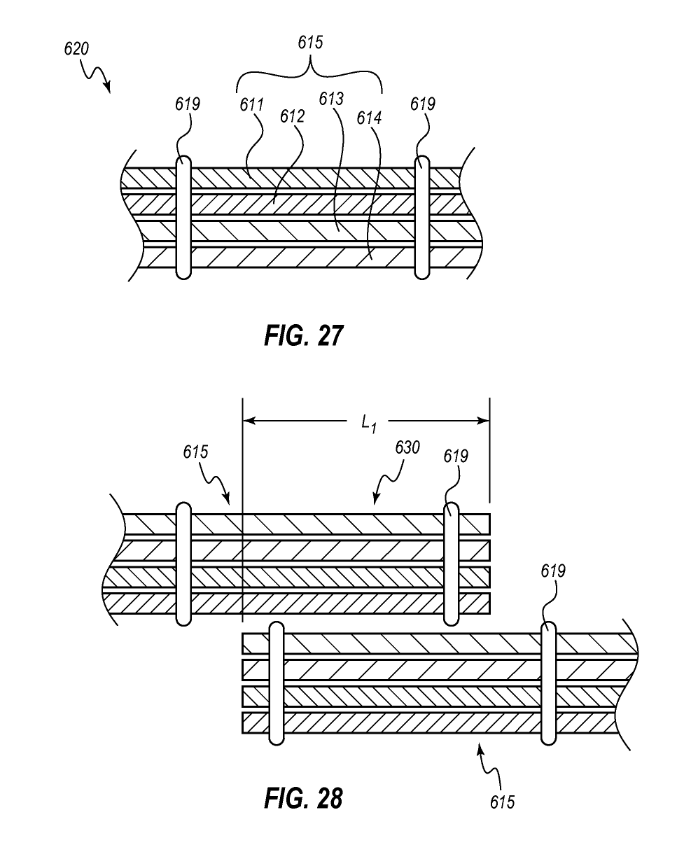

FIG. 27 illustrates a flexible four-layer composite construction 620, such as that described above with respect to FIG. 23B. Seams such as described hereafter may be used with composite constructions 620 having more or fewer layers.

FIG. 28 illustrates a flexible seam 630 that uses overlapping regions of ballistic-resistant armor 615 that slide relative to one another but do not allow a vulnerable path through the material. The dexterity seam 630 can have an overlap length L₁ of 0.1 in to 6 inches that will change based on the hip joint position but an overlap length will remain during most common placements of the wearer's hip position. A width or region of any of the seams discussed herein can be within a range of from about 0.1 to about 6 inches, or can be no greater than about 0.1, 0.2, 0.3, 0.5, 0.75, or 1 inches.

FIG. 29 illustrates another embodiment of a flexible seam 631 in the ballistic resistant armor 615. Instead of carrying all for layers across the seam 631, folds 641, 642 are placed in the layers and secured with stitching 619. A gap between the folds 641, 642 may be relatively narrow, and in some cases the folds 641, 642 overlap each other. In various embodiments, the gap is no greater than about 0.1, 0.2, or 0.25 inches. The folds 641, 642 may extend along fold lines. In the illustrated embodiment, the fold lines are not shown, as they extend in a direction perpendicular to the plane of the page. The upper two layers of the composite material 615 are discontinuous at the seam 631. The upper two layers of a portion of the composite material 615 that extend to the left of the seam 631 are formed by a single sheet of anti-ballistic material that is folded over on itself. Likewise, the upper two layers of the portion of the composite material 615 that extends to the right of the seam 631 are formed by a single sheet of anti-ballistic material that is folded over on itself.

FIG. 30 illustrates another embodiment of a flexible seam 632 in a ballistic resistant armor 615. Instead of carrying all for layers across a flexing region of the material, folds 651, 652 are placed in the upper layers, which are secured with stitching 619. Moreover, the lowest layer of anti-ballistic material is discontinuous at the seam 632. Two separate pieces of material 653, 654 either abut one another, slightly overlap each other, or are spaced a small distance apart from each other at the seam 632. Any gap between the folds 651, 652 and any gap between the adjacent lower layers 653, 654 can desirably be small. In various embodiments, each of the gaps is no greater than about 0.1, 0.2, or 0.25 inches across.

FIG. 31 illustrates another embodiment of a flexible seam 633 in the ballistic resistant armor 615. The seam 633 can resemble the seam 631 described above, except that folds 661, 662 are formed in the intermediate layers, rather than the upper layers, and the folds 661, 662 are sandwiched between continuous sheets of ballistic-resistant material.

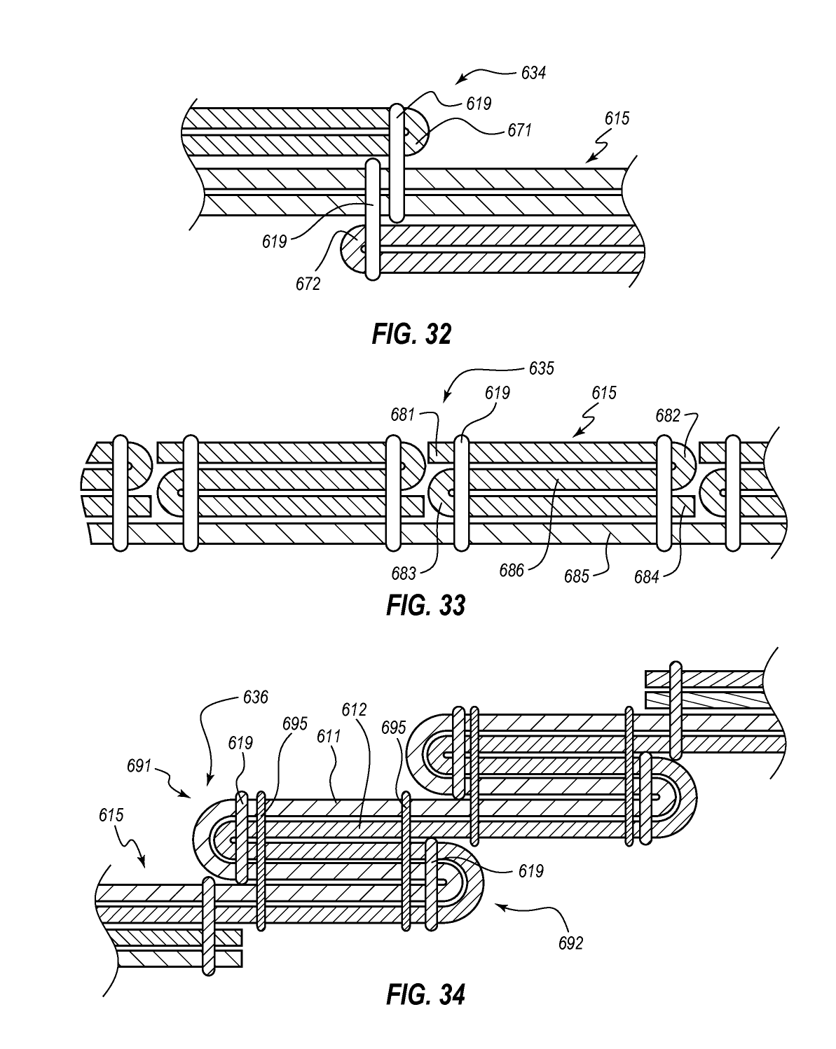

FIG. 32 illustrates another embodiment of a flexible seam 634 in the ballistic resistant armor 615. Instead of carrying all four layers across a flexing region of the material, folds 671, 672 are placed in the upper two and lower two layers, respectively, and are secured with stitching 619. The folded materials are separated by two layers of continuous material. The upper two and lower two layers overlap in the region of the seam 634 such that four layers of ballistic-resistant material are present on either side of the seam 634, and six layers of ballistic-resistant material are present in the region of the seam 634. It may be said that, in the region of the seam 634, a line that is perpendicular to an outer surface of the uppermost layer of ballistic-resistant material passes through at least four layers of ballistic-resistant material. In this embodiment, the line passes through six layers.

FIG. 33 illustrates another embodiment of a flexible seams 635 in a composite construction in the ballistic resistant armor 615. Instead of carrying all for layers across the seam 635, folds 682, 683 are placed in a single layer 686 of material, that the material is secured with stitching 619 to a base layer 685. The base layer 685 provides the material with flexibility and the folded layer 686 increases the protection level to the wearer.

FIG. 34 illustrates another embodiment of a flexible seam 636 in a composite construction of the ballistic resistant armor 615. Instead of carrying all for layers across a flexing region of the material, folds 691, 692 are placed in folded layers 611, 612 and secured with stitching 619 back onto themselves. A second resiliently deformable elastic stitching 695 allows the folds to open up like an accordion and then retract with changes in the wearer's anatomy.

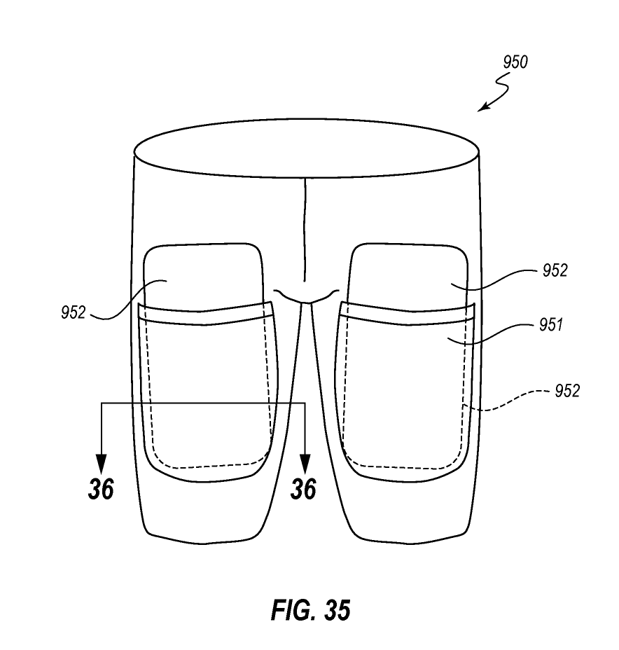

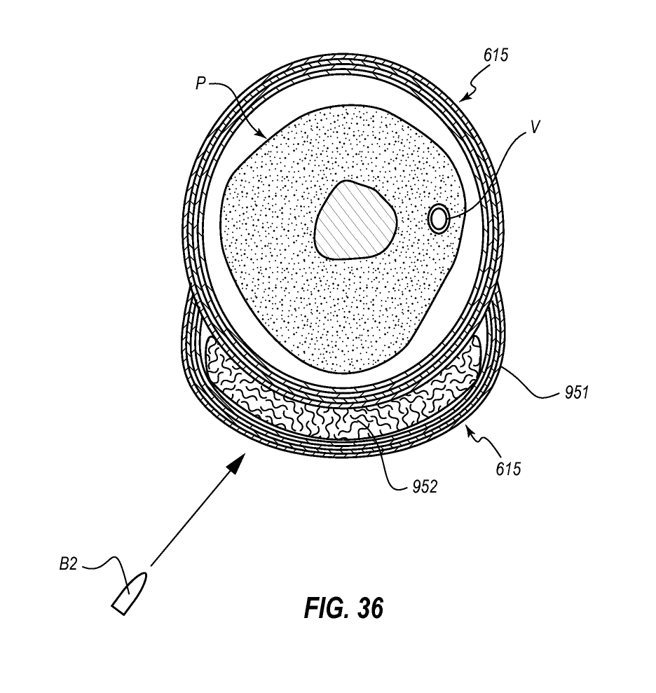

FIGS. 35-36 illustrate an embodiment of a ballistic-resistant shield 950 that can be integrated into any suitable embodiment of tactical pants described herein. Kinetic panels 952 are placed in pockets or openings configured in the ballistic resistant garment 950. The panels 952 act to disburse the kinetic energy of a projectile 82. The kinetic panels 852 may be constructed of a dense but lightweight material, such as, for example, carbon fiber, and can be secured to a flexible anti-ballistic layer (e.g., an aramid blend) via stitching, adhesive, etc. In other embodiments, the kinetic panels 852 may remain freely detached from the flexible anti-ballistic layer, and may be placed in pocket-like openings constructed in the ballistic-resistant shield 950.

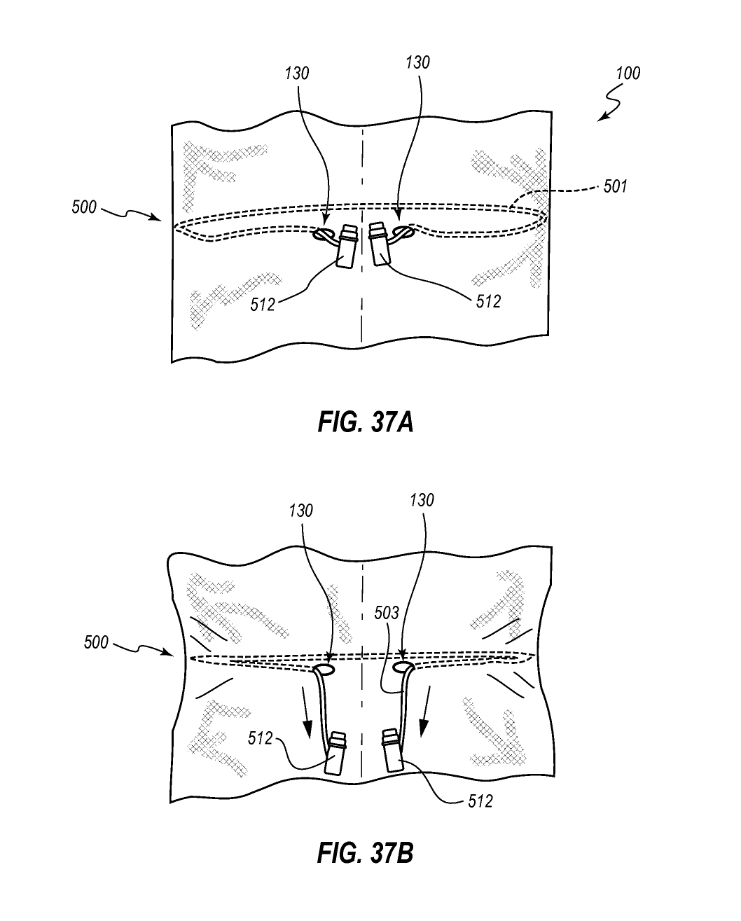

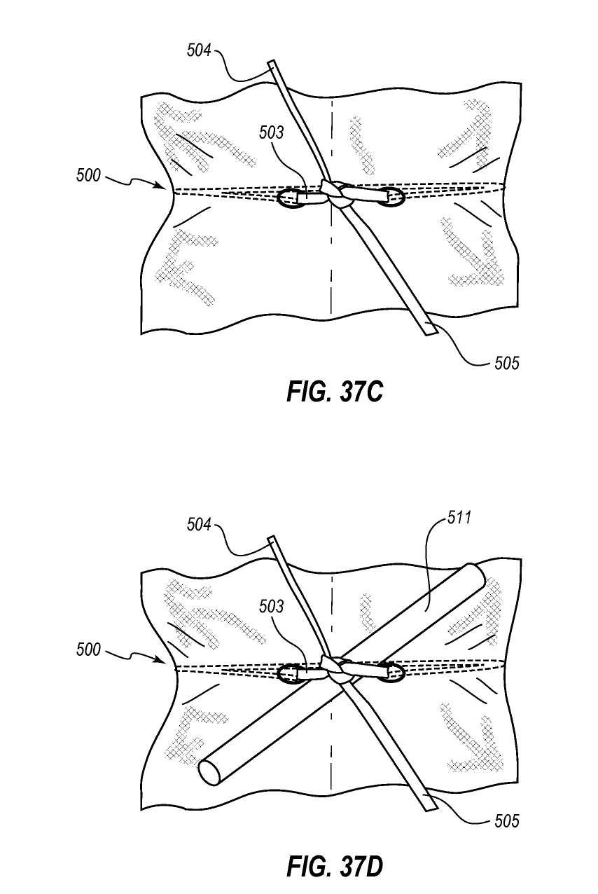

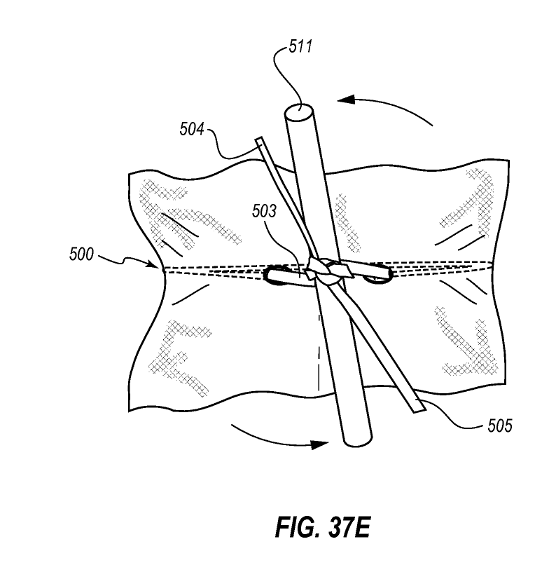

FIGS. 37A-37E illustrate an embodiment of a tourniquet assembly 500 that is integrated with a pair of tactical pants 100. The tourniquet assembly 500 can resemble either of the tourniquet assemblies 500u, 500l discussed above. Tourniquets at the knee and shin areas may be strategically located relative to the wearer's anatomy in special compartments of the tactical pants. Upon sustaining an injury the user can deploy the tourniquet 500 immediately. In some embodiments the tourniquet assemblies 500 are located approximately three inches above and three inches below the knee. A tourniquet member 501, such as a cord, may have an extremely high strength capacity. As shown in the method stages depicted in FIGS. 37A-37E, the ends of the cord 501 can be pulled through the openings 130. A knot can be tied in the ends of the cord 501. A torque rod 511 can be inserted behind the knot and then rotated to twist the knotted cord and thereby put tension on cord 501. The torque rod 511 can then be secured in place until further medical assistance can be obtained.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “about” or “approximately.” For each such reference, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. For example, where the term “substantially rectangular” is recited with respect to a feature, it is understood that in further embodiments, the feature can have a precisely rectangular orientation.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of the preceding claims up to and including claim [x],” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claim 1, 2, or 3, with these separate dependencies yielding three distinct embodiments; claim 5 can depend from any one of claim 1, 2, 3, or 4, with these separate dependencies yielding four distinct embodiments; and so on.

Recitation in the claims of the term “first” with respect to a feature or element does not necessarily imply the existence of a second or additional such feature or element. Elements specifically recited in means-plus-function format, if any, are intended to be construed in accordance with 35 U.S.C. §112¶6. Embodiments of the invention in which an exclusive property or privilege is claimed are defined as follows.

1. A pair of pants, comprising:
a waist portion configured to be worn around a waist of a wearer of the pants so as to support the pants thereon;
a leg portion extending from the waist portion;
a utility module attached to the leg portion, wherein the utility module is configured to retain a utility object; and
a harness that comprises a longitudinal strap system coupled with each of the utility module and the waist portion, wherein at least a portion of the longitudinal strap system extends between the utility module and the waist portion so as to support at least a portion of the weight of the utility module and any utility object retained thereby, wherein a portion of the longitudinal strap system is unattached to the leg portion, and wherein a length of the portion of the longitudinal strap system that is unattached to the leg portion is adjustable.

2. The pants of claim 1, wherein the longitudinal strap system is coupled with the utility module at a first anchor region and is coupled with the waist portion at a second anchor region, and wherein the portion of the longitudinal strap system that is unattached to the leg portion extends between the first and second anchor regions.

3. The pants of claim 2, wherein the length of the portion of the longitudinal strap system that extends between the first and second anchor points is configured to be adjusted without adjusting a length of any neighboring region of the leg portion.

4. The pants of claim 1, wherein the longitudinal strap system comprises an upper strap and a lower strap that are configured to be selectively coupled with and decoupled from each other via one or more connectors.

5. The pants of claim 1, wherein the longitudinal strap system comprises a strap that extends through a tensioning member, and wherein the length of the longitudinal strap system is configured to be adjusted by advancing the strap through the tensioning member.

6. The pants of claim 1, wherein the longitudinal strap system extends over a top edge of the waist portion such that a first portion of the longitudinal strap system is at an exterior of the waist portion and a second portion of the longitudinal strap system is at an interior of the waist portion, and wherein the longitudinal strap system is coupled to the waist portion at an inner surface and an outer surface of the waist portion.

7. The pants of claim 6, wherein the longitudinal strap system is coupled to the waist portion via stitching that extends through each of the first portion of the longitudinal strap system, the waist portion, and the second portion of the longitudinal strap system.

8. The pants of claim 1, wherein the longitudinal strap system defines a belt loop such that the harness is configured to transfer weight from the utility module and any utility object retained thereby to a belt when the belt is positioned through the belt loop.

9. The pants of claim 1, wherein one or more of the waist portion and the leg portion comprises a first material, and wherein the longitudinal strap system comprises a strap formed of a second material that has one or more strength properties that are greater than that of the first material.

10. The pants of claim 1, wherein the longitudinal strap system is stitched to a region of the leg portion that is attached to the utility module.

11. The pants of claim 1, wherein the harness further comprises a lateral strap system that encompasses the utility module, wherein the lateral strap system is adjustable so as to support the utility module against a leg of a wearer of the pants at a desired tension.

12. The pants of claim 11, wherein the lateral strap system comprises an expansion region and a support region, wherein the expansion region is configured to resiliently expand and contract, and wherein the support region is less extensible than the expansion region.

13. The pants of claim 12, wherein the lateral strap system is configured to operate in a supporting mode or in a tourniquet mode,
wherein, in the supporting mode, the expansion region of the lateral strap system is configured to expand and contract as a perimeter of a leg of a wearer of the pants expands and contracts due to muscular activity, such that the lateral strap system supports the utility module against the leg within a range of tensions, and
wherein, in the tourniquet mode, the lateral strap system is at a substantially fixed tension that is greater than any tension within the range of tensions of the supporting mode.

14. The pants of claim 1, wherein the utility module comprises a pair of brackets that are configured to be selectively coupled with and decoupled from each other.

15. The pants of claim 1, further comprising one or more layers of ballistic-resistant material positioned in the leg portion so as shield a femoral artery of a wearer of the pants.

16. The pants of claim 15, wherein at least a portion of the longitudinal strap system is at an interior of a region defined by the ballistic-resistant material so as to be readily accessible by a wearer of the pants.

17. A pair of pants, comprising:
a waist portion;
a leg portion extending from the waist portion;
a utility module attached to the leg portion, wherein the utility module is configured to retain a utility object; and
a harness that comprises a lateral strap system that encompasses the utility module, wherein the lateral strap system is adjustable so as to support the utility module against a leg of a wearer of the pants at a desired tension, wherein the lateral strap system further comprises an expansion region and a support region, wherein the expansion region is configured to resiliently expand and contract, and wherein the support region is less extensible than the expansion region.

18. The pants of claim 17, wherein the lateral strap system is configured to operate in a supporting mode or in a tourniquet mode,
wherein, in the supporting mode, the expansion region of the lateral strap system is configured to expand and contract as a perimeter of a leg of a wearer of the pants expands and contracts due to muscular activity such that the lateral strap system supports the utility module against the leg within a range of tensions, and
wherein, in the tourniquet mode, the lateral strap system is at a substantially fixed tension that is greater than any tension that is within the range of tensions.

19. The pants of claim 18, wherein the expandable region is taut and substantially inextensible when the lateral strap system operates in the tourniquet mode.

20. The pants of claim 18, wherein the lateral strap system comprises a locking assembly that is configured to assist a wearer of the pants in tightening the lateral strap system so as to achieve the substantially fixed tension and is configured to maintain the substantially fixed tension.

21. The pants of claim 17, wherein at least a portion of the lateral strap system that is at an interior of the leg portion is unattached to the leg portion such that a perimeter of the support region can be adjusted without adjusting a perimeter of a neighboring region of the leg portion.

22. The pants of claim 21, wherein a portion of the lateral strap system is at an exterior of the leg portion so as to be readily accessible by a wearer of the pants to adjust the perimeter of the support region.

23. The pants of claim 17, wherein the utility module comprises a first bracket attached to the leg portion and a second bracket that is configured to be selectively coupled with and decoupled from the first bracket.

24. The pants of claim 17, wherein the harness further comprises a longitudinal strap system that is configured to transfer at least a portion of the weight of the utility module and any utility object retained thereby to the waist portion of the pants.

25. The pants of claim 17, further comprising one or more layers of ballistic-resistant material positioned in the leg portion so as shield a femoral artery of a wearer of the pants.

26. The pants of claim 25, wherein the lateral strap system extends through the ballistic-resistant material.

27. A pair of pants, comprising:
a material arranged so as to form a leg of the pants that is elongated in a longitudinal direction; and
a tourniquet member that extends through at least a portion of the material that forms the leg of the pants so as to be maintained at a fixed longitudinal position relative to the leg of the pants, wherein the tourniquet member is configured to encompass at least a portion of a leg of the wearer of the pants.

28. The pants of claim 27, wherein the tourniquet member is configured to be tensioned so as to reduce blood flow through a femoral artery of a wearer of the pants without removing any portion of the leg of the pants from the wearer.

29. The pants of claim 27, further comprising a tensioning device that is configured to apply an adjustable tension to the tourniquet member.

30. The pants of claim 29, wherein the tensioning device comprises a locking assembly that is configured to grip the tourniquet member and tighten the tourniquet member as a lever is rotated.

31. The pants of claim 29, wherein the tensioning device comprises a torque rod that is contained in and is removable from a compartment of the pants.

32. The pants of claim 27, wherein the tourniquet member comprises a cord that extends through a compartment that is at least partially defined by the leg material such that opposite ends of the cord are at an exterior of the compartment, wherein the compartment extends about at least a majority of a perimeter of the leg of the pants.

33. The pants of claim 32, wherein at least a portion of the compartment is defined by stitching.

34. The pants of claim 27, wherein the tourniquet member comprises a strap that extends into and out of an interior of the leg of the pants via separate openings in the material, wherein the portion of the strap that is at the interior of the leg is unattached to the leg such that a perimeter of the strap can be adjusted without adjusting a perimeter of leg.

35. The pants of claim 27, wherein the leg of the pants comprises a ballistic-resistant material, and wherein the tourniquet member extends through the ballistic-resistant material.

36. A pair of pants, comprising:
a material arranged so as to form a leg of the pants that is elongated in a longitudinal direction;
a first tourniquet member that extends through at least a portion of the material that forms the leg of the pants so as to be maintained at a first fixed longitudinal position relative to the leg of the pants; and
a second tourniquet member that extends through at least a portion of the material that forms the leg of the pants so as to be maintained at a second fixed longitudinal position relative to the leg of the pants that is spaced from the first fixed longitudinal position.

37. The pants of claim 36, wherein the first fixed longitudinal position is at an upper leg region and the second fixed longitudinal position is at a lower leg region of the leg of the pants.

38. The pants of claim 36, wherein the leg of the pants comprises a ballistic-resistant material, and wherein at least the first tourniquet member extends through the ballistic-resistant material.

39. A pair of pants comprising:
a flexible material defining at least a waist region and a leg region that extends from the waist region; and
a ballistic-resistant shield coupled to the flexible material and positioned at an interior of at least a portion of the leg region, wherein the ballistic-resistant shield covers at least one quarter of an internal perimeter along a portion of a length of the leg region so as to extend about at least a portion of a femoral artery of a wearer of the pants, wherein the ballistic-resistant shield comprises:
a plurality of layers of ballistic-resistant material stitched together into one or more composite materials; and
a seam at which one or more composite materials meet, wherein at least one layer of a ballistic-resistant material is discontinuous at the seam so as not to continue from one side of the seam to another side of the seam, such that bending of the ballistic-resistant shield is easier at the seam than it is at regions of the one or more composite materials that are adjacent to the seam.

40. The pants of claim 39, wherein the ballistic-resistant shield covers a full internal perimeter along a portion of a length of the leg portion so as to fully encompass a femoral artery of a wearer of the pants along at least a portion of the length of the femoral artery.

41. The pants of claim 39, wherein the seam is at a junction of a waist portion and a leg portion of the shield.

42. The pants of claim 39, wherein at least one layer of ballistic-resistant material is continuous through the seam so as to pass from one side of the seam the another side of the seam without interruption.

43. The pants of claim 39, wherein the ballistic-resistant shield comprises a composite material that comprises at least two layers formed from a single piece of ballistic-resistant material, wherein the single piece of ballistic-resistant material is folded along a fold line, and wherein the fold line is positioned at the seam.

44. The pants of claim 39, wherein at least four layers of ballistic-resistant material are present at the seam such that a line perpendicular to an outer surface of the outermost layer of ballistic-resistant material passes through at least four layers of ballistic-resistant material.

45. The pants of claim 39, wherein a first composite material overlaps a second composite material at the seam such that the first composite material is configured to move over the second composite material as a wearer of the pants moves, and such that a width of the seam varies with movement of a wearer of the pants.

46. The pants of claim 39, at least one layer of the plurality of layers of ballistic-resistant material comprises woven aramid fiber.

47. The pants of claim 39, wherein the plurality of layers of ballistic-resistant material comprises at least four layers of aramid woven fiber.

48. The pants of claim 39, wherein ballistic-resistant shield is sufficiently ballistic resistant to provide ballistic protection of at least level IIA, as classified by the National Institute of Justice Standard 0101.06 of July 2008, such that the shield can protect a femoral artery of a wearer of the pants from 9 millimeter Full Metal Jacketed Round Nose bullets weighing 8.0 grams and moving at 373 meters per second, plus or minus 9.1 meters per second.

49. The pants of claim 39, further comprising a kinetic panel positioned to protect a femoral artery of a wearer of the pants.

50. The pants of claim 49, wherein the kinetic panel is coupled with the ballistic-resistant shield.
